SEQUENCE LISTING (6 sequences)

SEQ ID NO: 1 - RNA, 123 nt, Arabidopsis thaliana
  source 1..123: mol_type = genomic RNA
  ncRNA 21..40: ncRNA_class = miRNA
caagagaaacgcaaagaaactgacagaagagagtgagcacacaaaggcaatttgcatatcattgcacttgcttctcttgcgtgctcactgctctttctgtcagattccggtgctgatctcttt

SEQ ID NO: 2 - RNA, 93 nt, Theobroma cacao
  source 1..93: mol_type = genomic RNA
  ncRNA 7..26: ncRNA_class = miRNA
agagactgacagaagagagtgagcacacgcaggcaattgtatgaaaagccatgcctttgcgggtgcgtgctcacttctcttctgtcagcttcg

SEQ ID NO: 3 - RNA, 120 nt, Theobroma cacao
  source 1..120: mol_type = genomic RNA
  ncRNA 21..40: ncRNA_class = miRNA
cattcttgggacatagaaattgacagaagagagtgagcacacagaggcacttgtataagtctatacttttgcttttgcgtgctcacttctctttctgtcagcttccagtgccggaatttg

SEQ ID NO: 4 - RNA, 113 nt, Malus domestica
  source 1..113: mol_type = genomic RNA
  ncRNA 15..34: ncRNA_class = miRNA
agggtaaaggacggtgacagaagagagtgagcacacatggtattttcttgcatgctgggttcatgcttgaagctttgtgtgcttactctctatctgtccacccacctctctct

SEQ ID NO: 5 - RNA, 143 nt, Citrus X sinensis
  source 1..143: mol_type = genomic RNA
  ncRNA 31..50: ncRNA_class = miRNA
ttctcattctaattcttggaacattgaaattgacagaagagagtgagcacacagaggcagttgtataattgtatacatgttgcttttgcgtgctcactgctctttctgtcagcttccagtaccggaatctgacttggccctct

SEQ ID NO: 6 - RNA, 113 nt, Mangifera indica
  source 1..113: mol_type = genomic RNA
  ncRNA 18..37: ncRNA_class = miRNA
tcttggaacatagaaattgacagaagagagtgagcacacagaggcacttgtataagatatatttttgcttttgcgtgctcacttctcgttctgtcagcttccagtgccgggat

(19) United States
(12) Patent Application Publication
(10) Pub. No.: US 2025/0234827 A1
(43) Pub. Date: Jul. 24, 2025

(54) CONTROLLING JUVENILE TO REPRODUCTIVE PHASE TRANSITION IN TREE CROPS

(71) Applicant: BETTERSEEDS LTD, Givat Chen (IL)

(72) Inventors: Tal Sherman, Rechovot (IL); Ido Margalit, Gan Yavne (IL); Shira Corem, Sdei Hemed (IL)

(21) Appl. No.: 18/843,468

(22) PCT Filed: Mar. 30, 2023
(86) PCT No.: PCT/IL2023/050335

§ 371 Date: Sep. 3, 2024

Related U.S. Application Data
(60) Provisional application No. 63/362,978, filed on Apr. 14, 2022.

(51) Int. Cl. A01H 1/00; A01H 1/06; A01H 5/12; C12N 5/04; C12N 9/22; C12N 15/52

(52) U.S. Cl. CPC A01H 1/121; A01H 1/06; A01H 5/12; C12N 5/04; C12N 9/222; C12N 15/52

(57) ABSTRACT

A method for controlling juvenile to adult phase transition in a tree plant is provided. The method comprises introducing using targeted genome editing, at least one genomic modification in an endogenous microRNA156 (miRNA156). A tree plant, plant cell, plant tissue, plant part or a seed is produced by the aforementioned method.

### CROSS-REFERENCE TO RELATED APPLICATIONS

This application is a 35 U.S.C. § 371 National Phase of International Application No. PCT/IL2023/050335, filed Mar. 30, 2023, which claims the benefit of priority from U.S. Provisional Patent Application No. 63/362,978, filed on Apr. 14, 2022, each which is hereby incorporated by reference in its entirety.

### SEQUENCE LISTING

The contents of the electronic sequence listing (501048-00002.xml; Size: 8,961 bytes; and Date of Creation: Aug. 29, 2024) is herein incorporated by reference in its entirety.

### FIELD OF THE INVENTION

The present disclosure relates to methods for modulating the duration of the juvenile phase of growth of plants and to plants produced by these methods.

### BACKGROUND OF THE INVENTION

The plant life cycle encompasses several developmental transitions, including the transition from an embryonic to post-embryonic mode of growth, the vegetative phase change (juvenile-to-adult vegetative transition), and the floral transition (vegetative-to-reproductive transition)—all of which involve temporal and spatially coordinated changes in various traits essential for plant survival and reproduction.

MicroRNAs play an important role in regulating the timing of plant developmental transitions. By regulating transcripts of developmental genes, miRNAs control some aspects of leaf morphology, polarity and floral organ identity, and some stress responses (Willmann and Poethig, Curr. Opin. Plant Biol. 8:548-552, 2005) as well as the timing of juvenile to adult vegetative phase change.

microRNA156 (miR156) is one of the most evolutionally conserved microRNAs in plants and has broad functions including regulation of stem, flower, and leaf development in various species (Schwab, et al., Dev. Cell 8:517-527, 2005; Xie, et al., Plant Physiol, 142:280-293, 2006; Fu, et al., Plant Biotechnol. J. 10:443-452, 2012; Chuck, et al., Proc. Natl. Acad. Sci. USA 108:17550-17555, 2011; Tatematsu, et al., Plant J. 82:596-608, 2015). Overexpression of miR156 dramatically improved tillering in rice (Xie, et al., 2006) and switchgrass (Fu, et al., 2012; Chuck, et al., 2011). miR156 targets SQUAMOSA PROMOTER BINDING PROTEIN LIKE (SPL) transcription factor genes (Reinhart, et al., Genes Dev. 16:1616-1626, 2002; Xing, et al., Plant Cell 22:3935-3950, 2010). It was reported that higher levels of OsSPL14 suppressed tillering while promoting panicle branching in rice (Jiao, et al., Nat. Genetics 42:541-544, 2010; Miura, et al., Nat. Genetics 42:545-549, 2010).

It was further reported that the maize and Arabidopsis signaling pathway and miRNA expression cascade are similar (Nonogaki, Plant Cell Physiol. 51:1840-1846, 2010). miR156 targets SBP-domain transcription factors—teosinte glume architecture1 (tga1) in maize and SPL13 in Arabidopsis. SPB transcription factors up regulate miR172 in both species and miR172 targets AP2-like transcription factors such as glossy15 in maize and SCHNARCHSAPFEN (SNZ) in Arabidopsis. In Cg1 mutants of maize, the overexpression of miR156 causes a decrease in tga1 and miR172, which cause an increase in expression of Glossy15.

Enhancement of biomass productivity of important agricultural and biofuel crops is highly desirable. Accordingly, the vegetative phase change is targeted by plant breeders to shorten the breeding cycle. However, there is very limited information on phase transition in economically important horticultural tree crops, which have a significantly long vegetative phase affecting fruit bearing.

In view of the above there is still a long felt and unmet need to develop methods for controlling juvenility and phase transition in tree crops.

### SUMMARY OF THE INVENTION

It is therefore one object of the present invention to disclose a method for controlling juvenile to adult phase transition in a tree plant, the method comprising introducing using targeted genome editing, at least one genomic modification in an endogenous microRNA156 (miRNA156).

It is a further object of the present invention to disclose the method as defined above, wherein the method comprises upregulation of a downstream gene target of the miRNA156.

It is a further object of the present invention to disclose the method as defined in any of the above, wherein the method comprises expressing in the tree plant a nuclease comprising a nucleic acid binding domain, wherein the nucleic acid binding domain binds to a target site in the miRNA156, to generate the genomic modification.

It is a further object of the present invention to disclose the method as defined in any of the above, wherein the nuclease is selected from CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease, or any combination thereof.

It is a further object of the present invention to disclose the method as defined in any of the above, wherein the nuclease introduces a mutation in the miRNA156 via a DNA cleavage domain that cleaves the miRNA156 encoding gene, or via base editing of the miRNA156 encoding gene.

It is a further object of the present invention to disclose the method as defined in any of the above, wherein the method comprises expressing in the tree plant a guide RNA nucleic acid that binds to a target site in the miRNA156, and a CRISPR-Cas endonuclease that associates with the gRNA nucleic acid.

It is a further object of the present invention to disclose the method as defined in any of the above, wherein said genomic modification is generated via introduction of (a) Cas DNA or a nuclease-deactivated (dCas) and gRNA polynucleotide comprising a nucleic acid sequence complementary to a target site in the miRNA156, or (b) a ribonucleoprotein (RNP) complex comprising Cas protein and gRNA polynucleotide comprising a nucleic acid sequence complementary to a target site in the miRNA156, and any combination thereof.

It is a further object of the present invention to disclose the method as defined in any of the above, wherein said gRNA sequence comprises a 3′ Protospacer Adjacent Motif (PAM) selected from the group consisting of NGG (SpCas), NNNNGATT (NmeCas9), NNAGAAW (StCas9), NAAAAC (TdCas9), NNGRRT (SaCas9), TTTR PAM (archaea Cas12f), and TBN (Cas-phi).

It is a further object of the present invention to disclose the method as defined in any of the above, wherein said Cas gene is selected from the group consisting of Cas3, Cas4, Cas5, Cas5e (or CasD), Cas6, Cas6e, Cas6f, Cas7, Cas8al, Cas8a2, Cas8b, Cas8c, Cas9, Cas10, Cast10d, Cas12, Cas13, Cas14, CasX, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, and Cu1966, bacteriophages Cas such as CasΦ (Cas-phi), split Cas such as split Cas12a or split Cas9 or nuclease-deactivated (dCas) and any combination thereof.

It is a further object of the present invention to disclose the method as defined in any of the above, wherein said tree plant comprises a DNA construct, said DNA construct comprising a promoter operably linked to a nucleotide sequence encoding a plant optimized Cas endonuclease, said plant optimized Cas endonuclease is capable of binding to and creating a double strand break in a genomic target sequence of said plant genome.

It is a further object of the present invention to disclose the method as defined in any of the above, wherein said genomic modification is a silencing mutation, a knockdown mutation, a knockout mutation, a loss of function mutation or any combination thereof.

It is a further object of the present invention to disclose the method as defined in any of the above, wherein said genomic modification is an insertion, deletion, indel or a substitution.

It is a further object of the present invention to disclose the method as defined in any of the above, wherein said genomic modification is an induced mutation in the coding region of said gene, in a conserved region within the gene, a mutation in the regulatory region of said gene, a mutation in a gene downstream or upstream of miRNA156, and/or an epigenetic factor.

It is a further object of the present invention to disclose the method as defined in any of the above, wherein said method comprises reducing the duration of juvenile phase in the tree plant by introduction of a targeted genomic modification in the microRNA156 (miRNA156), thereby down regulating expression of the endogenous microRNA156 (miRNA156) and/or upregulating of a downstream gene target of the miRNA156.

It is a further object of the present invention to disclose the method as defined in any of the above, wherein the downstream gene target of the miRNA156 is selected from genes comprising a miR156 recognition site or element.

It is a further object of the present invention to disclose the method as defined in any of the above, wherein the downstream gene target of the miRNA156 is selected from genes encoding SQUAMOSA PROMOTER BINDING PROTEIN (SBP)-domain containing transcription factors, SQUAMOSA PROMOTER BINDING PROTEIN-LIKE (SPL) transcription factors and SUPPRESSOR OF OVEREXPRESSION OF CONSTANS 1 (SOC1).

It is a further object of the present invention to disclose the method as defined in any of the above, wherein the tree plant is selected from horticultural tree crops such as fruit tree species including: Theobroma cacao (cacao), Mangifera indica (mango), Persea Americana (avocado), Macadamia integrifolia (macadamia), Prunus dulcis (almond), Malus domestica (apple), Prunus armeniaca (apricot), Averrhoa carambola (carambola, starfruit), Anacardium occidentale (cashew), Prunus avium (cherry), citrus (orange, lemon, lime, etc.), Cocos nucifera (coconut), Citrus x paradisi (grapefruit), Psidium guajava (guava), Litchi chinensis (lychee), Olea europaea (olive), Prunus persica (peach) and Prunus persica var. nucipersica (nectarine), Pyrus (pear), Carya illinoinensis (pecan), Diospyros kaki (persimmon), Prunus domestica (plum), Citrus maxima (pomelo), Punica granatum (pomegranate), Castanea sativa (sweet chestnut), Solanum betaceum (tamarillo) and Juglans (walnut).

It is a further object of the present invention to disclose the method as defined in any of the above, wherein the tree plant comprises enhanced productivity trait (fruit or vegetable yield), enhanced agronomic trait and/or enhanced plant biomass.

It is a further object of the present invention to disclose the method as defined in any of the above, wherein the tree crop exhibits time reduced juvenility or vegetative phase change, increased duration of adult or mature phase, and/or early transition into adult or mature or reproductive phase, relative to a corresponding tree plant lacking the targeted genomic modification.

It is a further object of the present invention to disclose the method as defined in any of the above, wherein the method comprises modulating the timing of juvenile to adult phase transition, the timing of juvenile to adult phase transition is calculated based on miRNA156 target genes expression level and is shorter compared to wild type non edited corresponding plants.

It is a further object of the present invention to disclose the method as defined in any of the above, wherein the tree plant exhibits an altered trait selected from the group consisting of: an altered proportion of juvenile, transitional, or adult leaves; enhanced yield of vegetative tissue; enhanced fruit yield, altered development or morphology, wherein the trait exhibited by the tree plant is altered relative to a corresponding wild type tree plant.

It is a further object of the present invention to disclose a method for controlling juvenile to adult phase transition in a tree plant, the method comprising using targeted genome editing, generating at least one genomic modification in an endogenous microRNA156 (miRNA156) and/or upregulating a downstream gene target of the miRNA156.

It is a further object of the present invention to disclose a tree plant, plant cell, plant tissue, plant part or a seed, produced by the method as defined in any of the above.

It is a further object of the present invention to disclose a tree plant exhibiting modulated timing of juvenile to adult phase transition, the tree plant comprises introduced targeted genome modification in a microRNA156 (miRNA156), and/or upregulated expression of a downstream gene target of the miRNA156.

It is a further object of the present invention to disclose the tree plant as defined in any of the above, wherein said tree plant exhibits time reduced juvenility or vegetative phase change, increased duration of adult or mature phase, and/or early transition into adult or mature or reproductive phase, relative to a corresponding tree plant lacking the targeted genomic modification.

It is a further object of the present invention to disclose the tree plant as defined in any of the above, wherein the tree plant is expressing a nuclease comprising a nucleic acid binding domain, wherein the nucleic acid binding domain binds to a target site in the miRNA156.

It is a further object of the present invention to disclose the tree plant as defined in any of the above, wherein the nuclease is selected from CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease, or any combination thereof.

It is a further object of the present invention to disclose the tree plant as defined in any of the above, wherein the nuclease introduces a mutation in the miRNA156 via a DNA cleavage domain that cleaves the miRNA156, or via base editing of the miRNA156.

It is a further object of the present invention to disclose the tree plant as defined in any of the above, wherein the tree plant is expressing a guide RNA nucleic acid that binds to a target site in the miRNA156, and a CRISPR-Cas endonuclease that associates with the gRNA nucleic acid.

It is a further object of the present invention to disclose the tree plant as defined in any of the above, wherein said genomic modification is generated via introduction of (a) Cas DNA and gRNA polynucleotide comprising a nucleic acid sequence complementary to a target site in the miRNA156, or (b) a ribonucleoprotein (RNP) complex comprising Cas and gRNA polynucleotide comprising a nucleic acid sequence complementary to a target site in the miRNA156, and any combination thereof.

It is a further object of the present invention to disclose the tree plant as defined in any of the above, wherein said gRNA sequence comprises a 3′ Protospacer Adjacent Motif (PAM) selected from the group consisting of NGG (SpCas), NNNNGATT (NmeCas9), NNAGAAW (StCas9), NAAAAC (TdCas9), NNGRRT (SaCas9), TTTR PAM (archaea Cas12f), and TBN (Cas-phi).

It is a further object of the present invention to disclose the tree plant as defined in any of the above, wherein said Cas gene is selected from the group consisting of Cas3, Cas4, Cas5, Cas5e (or CasD), Cas6, Cas6e, Cas6f, Cas7, Cas8a1, Cas8a2, Cas8b, Cas8c, Cas9, Cas10, Cast10d, Cas12, Cas13, Cas14, CasX, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, and Cu1966, bacteriophages Cas such as CasΦ (Cas-phi), split Cas such as split Cas12a or split Cas9 or nuclease-deactivated (dCas) and any combination thereof.

It is a further object of the present invention to disclose the tree plant as defined in any of the above, wherein said tree plant comprises a DNA construct, said DNA construct comprising a promoter operably linked to a nucleotide sequence encoding a plant optimized Cas endonuclease, said plant optimized Cas endonuclease is capable of binding to and creating a double strand break in a genomic target sequence of said plant genome.

It is a further object of the present invention to disclose the tree plant as defined in any of the above, wherein said genomic modification is a silencing mutation, a knockdown mutation, a knockout mutation, a loss of function mutation or any combination thereof.

It is a further object of the present invention to disclose the tree plant as defined in any of the above, wherein said genomic modification is an insertion, deletion, indel or a substitution.

It is a further object of the present invention to disclose the tree plant as defined in any of the above, wherein said genomic modification is an induced mutation in the coding region of said gene, in a conserved region within the gene, a mutation in the regulatory region of said gene, a mutation in a gene downstream or upstream of miRNA156, and/or an epigenetic factor.

It is a further object of the present invention to disclose the tree plant as defined in any of the above, wherein said tree plant exhibits reduced duration of the juvenile phase, further wherein the plant comprises a targeted genomic modification in the microRNA156 (miRNA156), said modification down regulate expression of the endogenous microRNA156 (miRNA156) and/or upregulate expression of a downstream gene target of the miRNA156.

It is a further object of the present invention to disclose the tree plant as defined in any of the above, wherein the downstream gene target of the miRNA156 is selected from genes comprising a miR156 recognition site or element.

It is a further object of the present invention to disclose the tree plant as defined in any of the above, wherein the downstream gene target of the miRNA156 is selected from genes encoding SQUAMOSA PROMOTER BINDING PROTEIN (SBP)-domain containing transcription factors, SQUAMOSA PROMOTER BINDING PROTEIN-LIKE (SPL) transcription factors and SUPPRESSOR OF OVEREXPRESSION OF CONSTANS 1 (SOC1).

It is a further object of the present invention to disclose the tree plant as defined in any of the above, wherein the tree plant is selected from horticultural tree crops such as fruit tree species including: Theobroma cacao (cacao), Mangifera indica (mango), Persea Americana (avocado), Macadamia integrifolia (macadamia), Prunus dulcis (almond), Malus domestica (apple), Prunus armeniaca (apricot), Averrhoa carambola (carambola, starfruit), Anacardium occidentale (cashew), Prunus avium (cherry), citrus (orange, lemon, lime, etc.), Cocos nucifera (coconut), Citrus x paradisi (grapefruit), Psidium guajava (guava), Litchi chinensis (lychee), Olea europaea (olive), Prunus persica (peach) and Prunus persica var. nucipersica (nectarine), Pyrus (pear), Carya illinoinensis (pecan), Diospyros kaki (persimmon), Prunus domestica (plum), Citrus maxima (pomelo), Punica granatum (pomegranate), Castanea sativa (sweet chestnut), Solanum betaceum (tamarillo) and Juglans (walnut).

It is a further object of the present invention to disclose the tree plant as defined in any of the above, wherein the tree plant comprises enhanced productivity trait (fruit or vegetable yield), enhanced agronomic trait and/or enhanced plant biomass.

It is a further object of the present invention to disclose the tree plant as defined in any of the above, wherein the timing of juvenile to adult phase transition is shorter compared to wild type non edited corresponding plants.

It is a further object of the present invention to disclose the tree plant as defined in any of the above, wherein the tree plant exhibits an altered trait selected from the group consisting of: an altered proportion of juvenile, transitional, or adult leaves; enhanced yield of vegetative tissue; enhanced fruit yield, altered development or morphology, wherein the trait exhibited by the tree plant is altered relative to a corresponding wild type tree plant.

It is a further object of the present invention to disclose a plant cell, plant tissue, regenerable cells, plant part or a seed of the tree plant as defined in any of the above.

It is a further object of the present invention to disclose a polynucleotide molecule comprising a promoter operably linked to a nucleotide sequence encoding a Cas nuclease and a guide RNA (gRNA) comprising a nucleic acid sequence complementary to a target site in miRNA156.

It is a further object of the present invention to disclose the polynucleotide molecule as defined above, wherein the sequence of the miRNA156 is selected from SEQ ID NO:1-6, a functional variant thereof or any combination thereof.

It is a further object of the present invention to disclose a recombinant vector comprising the polynucleotide molecule as defined in any of the above.

It is a further object of the present invention to disclose the recombinant vector as defined in any of the above, further comprising at least one additional sequence selected from: a regulatory sequence, a selectable marker, a leader sequence and a terminator.

### BRIEF DESCRIPTION OF THE FIGURES

Exemplary non-limited embodiments of the disclosed subject matter will be described, with reference to the following description of the embodiments, in conjunction with the figures.

The figures are generally not shown to scale and any sizes are only meant to be exemplary and not necessarily limiting. Corresponding or like elements are optionally designated by the same numerals or letters.

FIG. 1 is presenting a sequence alignment of microRNA156 (miR156) precursors from various tree crop species. The box presented highlights the mature miR156 sequence region.

FIG. 2 is presenting Arabidopsis thaliana miR156a (At miR156a) sequence as set forth in SEQ ID NO:1;

FIG. 3 is presenting Theobroma cacao miR156b (Tc miR156b) sequence as set forth in SEQ ID NO:2;

FIG. 4 is presenting Theobroma cacao miR156a (Tc miR156a) sequence as set forth in SEQ ID NO:3;

FIG. 5 is presenting Malus domestica miR156m (Md miR156m) sequence as set forth in SEQ ID NO:4;

FIG. 6 is presenting Citrus x sinensis miR156b (Cs miR156b) sequence as set forth in SEQ ID NO:5;

FIG. 7 is presenting Mangifera indica miR156 (Mi miR156) sequence as set forth in SEQ ID NO:6;

FIG. 8 is presenting secondary structure prediction of At miR156a;

FIG. 9 is presenting secondary structure prediction of Tc miR156b;

FIG. 10 is presenting secondary structure prediction of Tc miR156a;

FIG. 11 is presenting secondary structure prediction of Md miR156m;

FIG. 12 is presenting secondary structure prediction of Cs miR156b; and

FIG. 13 is presenting secondary structure prediction of Mi miR156.

### DETAILED DESCRIPTION OF THE PREFERRED EMBODIMENTS

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured.

The present invention provides a solution for the need to shorten juvenile to reproductive transition time period in order to allow enhanced plant biomass production.

The present invention provides a method for controlling juvenile to adult phase transition in a tree plant. The method comprises introducing using targeted genome editing, at least one genomic modification in an endogenous microRNA156 (miRNA156).

The aforementioned method further comprises upregulation of a downstream gene target of the miRNA156, e.g. by the downregulation of miRNA156 using targeted gene editing and thereby upregulating expression of genes in the pathway downstream of miRNA156.

The present invention further provides a tree plant or any part thereof, such as a cell or a seed, with altered juvenile to reproductive transition time period, produced using genome editing or other genome modification techniques.

According to one embodiment, a tree plant exhibiting modulated timing of juvenile to adult phase transition is herein provided. The tree plant comprises introduced targeted genome modification in a microRNA156 (miRNA156), or upregulated expression of a downstream gene target of the miRNA156.

It is herein acknowledged that in plants, the transition from juvenile to reproductive phase is controlled by various regulatory networks. One of these networks is the microRNA pathway. Two well-known miRNA's controlling this process are miR156 and miR172. While miRNA156 is highly expressed during the juvenile phase, its expression decreases while transitioning into the mature phase. On the other hand, miRNA172 levels are low during the juvenile phase and increases while entering the mature phase (miR156 and miR172 are expressed in inverse patterns) (Wu, G. et al. The Sequential Action of miR156 and miR172 Regulates Developmental Timing in Arabidopsis. Cell 138, 750-759, 2009). It has been shown that miRNA156 controls expression levels of the SQUAMOSA-PROMOTER BINDING PROTEIN-LIKE (SPL) genes in apple (Jia, X. L. et al. miR156 switches on vegetative phase change under the regulation of redox signals in apple seedlings. Sci. Rep. 7, 14223, 2017), as well as in avocado, macadamia and mango (Ahsan, M. U. et al. Juvenility and Vegetative Phase Transition in Tropical/Subtropical Tree Crops. Front. Plant Sci. 10, 729, 2019).

It is therefore one object of the present invention to disclose a modified plant (e.g. genome edited plant) exhibiting a shorter juvenile stage that transitions faster into the adult stage. This is achieved by editing the opposing miRNA156 and miR172 gene families and or downstream targets of these miRNAs.

It is within the scope of the current invention that the microRNA156 (miR156) regulates the vegetative phase change via inhibition of the target SQUAMOSA-PROMOTER BINDING PROTEIN-LIKE (SPL) genes.

The present invention shows that the miRNAs (e.g. miR156) can be successfully knocked-down by targeted genome editing using the CRISPR/Cas nuclease fused to gRNA-PAM, complementary to miR156 sequence.

Up until now it was reported that during the vegetative phase change in apple, ROS accumulate and miR156 transcription declined. In Arabidopsis, it was published that miR156 targets 10 out of 16 SPL family members (SPL2, SPL3, SPL4, SPL5, SPL6, SPL9, SPL10, SPL11, SPL13, SPL15), all characterized by a 76-amino acid DNA-binding domain termed SBP (SQUAMOSA PROMOTER BINDING PROTEIN) (Birkenbihl et al., 2005; He et al., 2018).

In general, it is herein acknowledged that in plants, juvenile to adult phase transition is regulated by the sequential activity of two microRNAs: miR156 and miR172. A decline in miR156 and increase in miR172 abundance is associated with phase transition.

In horticultural tree crops, a long juvenile phase poses a serious hindrance to crop selection and improvement because breeding and productivity traits are dependent on attaining reproductive status (i.e., flowering and fruiting). However, there is limited information on phase transition in economically important horticultural tree crops.

The aim of the current invention is, for the first time, to provide a genome editing based method for controlling juvenile to adult phase transition in tree crops. Thus important horticultural trees (such as fruit bearing trees) with shortened juvenile to reproductive transition time period can be produced.

The microRNA156 control of the vegetative phase change in plants is under transcriptional regulation.

According to one embodiment, it is shown that miR156 transcript level knockdown correlates with enhancing juvenility phase transition in these tree crops. This could be achieved also by upregulation of a downstream gene target of the miRNA156, such as SPL genes, as miR156 expression anticorrelates with accumulation of SPL genes and other floral homeotic genes including AP1 and SOC1.

As used herein the term “about” denotes ±25% of the defined amount or measure or value.

As used herein the term “similar” denotes a correspondence or resemblance range of about ±20%, particularly ±15%, more particularly about ±10% and even more particularly about ±5%.

As used herein the term “corresponding” generally means similar, analogous, like, alike, akin, parallel, identical, resembling or comparable. In further aspects it means having or participating in the same relationship (such as type or species, kind, degree, position, correspondence, or function). It further means related or accompanying. In some embodiments of the present invention it refers to plants of the same species or strain or variety or to sibling plant, or one or more individuals having one or both parents in common.

The term “corresponding” or “corresponding to” or “corresponding to nucleotide sequence” or “corresponding to position” as used herein, also refers in the context of the present invention to sequence homology or sequence identity. These terms relate to two or more nucleic acid or protein sequences, that are the same or have a specified percentage of amino acid residues or nucleotides that are the same, when compared and aligned for maximum correspondence, as measured using one of the available sequence comparison algorithms or by visual inspection. If two sequences, which are to be compared with each other, differ in length, sequence identity preferably relates to the percentage of the nucleotide residues of the shorter sequence, which are identical with the nucleotide residues of the longer sequence. As used herein, the percent of identity or homology between two sequences is a function of the number of identical positions shared by the sequences, taking into account the number of gaps, and the length of each gap, which needs to be introduced for optimal alignment of the two sequences. The comparison of sequences and determination of identity percent between two sequences can be accomplished using a mathematical algorithm as known in the relevant art. According to further aspects of the invention, the term “corresponding to the nucleotide sequence” or “corresponding to position”, refers to variants, homologues and fragments of the indicated nucleotide sequence, which possess or perform the same biological function or correlates with the same phenotypic characteristic of the indicated nucleotide sequence.

Another indication that two nucleic acid sequences are substantially identical or that a sequence is “corresponding to the nucleotide sequence” is that the two molecules hybridize to each other under stringent conditions. High stringency conditions, such as high hybridization temperature and low salt in hybridization buffers, permits only hybridization between nucleic acid sequences that are highly similar, whereas low stringency conditions, such as lower temperature and high salt, allows hybridization when the sequences are less similar.

In other embodiments of the invention, such substantially identical sequences refer to polynucleotide or amino acid sequences that share at least about 80% similarity, preferably at least about 90% similarity, alternatively, about 95%, 96%, 97%, 98% or 99% similarity to the indicated polynucleotide or amino acid sequences.

According to other aspects of the invention, the term “corresponding” refers also to complementary sequences or base pairing such that when they are aligned antiparallel to each other, the nucleotide bases at each position in the sequences will be complementary. The degree of complementarity between two nucleic acid strands may vary.

A “plant” as used herein refers to any plant at any stage of development, particularly a seed plant. The term “plant” includes the whole plant or any parts or derivatives thereof, such as plant cells, seeds, plant protoplasts, plant cell tissue culture from which tomato plants can be regenerated, plant callus or calli, meristematic cells, microspores, embryos, immature embryos, pollen, ovules, anthers, fruit, flowers, leaves, cotyledons, pistil, seeds, seed coat, roots, root tips and the like.

The term “plant cell” used herein refers to a structural and physiological unit of a plant, comprising a protoplast and a cell wall. The plant cell may be in a form of an isolated single cell or a cultured cell, or as a part of higher organized unit such as, for example, plant tissue, a plant organ, or a whole plant.

The term “plant cell culture” as used herein means cultures of plant units such as, for example, protoplasts, regenerable cells, cell culture, cells, cells in plant tissues, pollen, pollen tubes, ovules, embryo sacs, zygotes and embryos at various stages of development, leaves, roots, root tips, anthers, meristematic cells, microspores, flowers, cotyledons, pistil, fruit, seeds, seed coat or any combination thereof.

The term “plant material” or “plant part” used herein refers to leaves, stems, roots, root tips, flowers or flower parts, fruits, pollen, egg cells, zygotes, seeds, seed coat, cuttings, cell or tissue cultures, or any other part or product of a plant or a combination thereof.

A “plant organ” as used herein means a distinct and visibly structured and differentiated part of a plant such as a root, stem, leaf, flower, flower bud, or embryo.

The term “Plant tissue” as used herein means a group of plant cells organized into a structural and functional unit. Any tissue of a plant in planta or in culture is included. This term includes, but is not limited to, whole plants, plant organs, plant seeds, tissue culture, protoplasts, meristematic cells, calli and any group of plant cells organized into structural and/or functional units. The use of this term in conjunction with, or in the absence of, any specific type of plant tissue as listed above or otherwise embraced by this definition is not intended to be exclusive of any other type of plant tissue.

As used herein, the term “progeny” or “progenies” refers in a non limiting manner to offspring or descendant plants. According to certain embodiments, the term “progeny” or “progenies” refers to plants developed or grown or produced from the disclosed or deposited seeds as detailed inter alia. The grown plants preferably have the desired traits of the disclosed or deposited seeds, i.e. reduced juvenility phase and enhanced adult phase of growth.

As used herein, the term “tree plant” or “tree crop” includes, in a non-limiting manner, fruit trees, citrus fruit trees, nut trees, berry plantations, vineyards, olive trees and other tree crops used for human consumption (e.g. tea, cacao or coffee) and for other purposes (e.g. nurseries). It includes perennial or annual plants. It further encompass orchards, vineyards and olive tree plantations.

Non limiting examples of tree crops, e.g. horticultural tree crops, such as fruit tree species within the scope of the present invention include: Theobroma cacao (cacao), Mangifera indica (mango), Persea Americana (avocado), Macadamia integrifolia (macadamia), Prunus dulcis (almond), Malus domestica (apple), Prunus armeniaca (apricot), Averrhoa carambola (carambola, starfruit), Anacardium occidentale (cashew), Prunus avium (cherry), citrus (orange, lemon, lime, etc.), Cocos nucifera (coconut), Citrus x paradisi (grapefruit), Psidium guajava (guava), Litchi chinensis (lychee), Olea europaea (olive), Prunus persica (peach) and Prunus persica var. nucipersica (nectarine), Pyrus (pear), Carya illinoinensis (pecan), Diospyros kaki (persimmon), Prunus domestica (plum), Citrus maxima (pomelo), Punica granatum (pomegranate), Castanea sativa (sweet chestnut), Solanum betaceum (tamarillo) and Juglans (walnut).

The term “microRNA” or “miRNA” refers hereinafter to small non-coding RNAs (including precursor and mature miRNA) that have been found in most of the eukaryotic organisms. They are involved in the regulation of gene expression at the post-transcriptional level in a sequence specific manner. In other words, microRNAs are small noncoding RNA molecules (20 to 24 nt) that negatively regulate eukaryotic gene activity post transcriptionally. MiRNAs are produced from their precursors by Dicer-dependent small RNA biogenesis pathway. MiRNAs are candidates for studying gene function using different RNA-based gene silencing techniques. For example, artificial miRNAs (amiRNAs) targeting one or several genes of interest is a potential tool in functional genomics.

In plants, each miRNA gene is transcribed by RNA polymerase II, thereby producing a pri-miRNA, which is then cleaved by dicer-like 1 (DCL1) and its accessory proteins serrate (SE) and hyponastic leaves (HYL1). This process leads to the formation of pre-miRNA followed by the release of the miRNA-5p/miRNA-3p duplex. The miRNA strand is selectively loaded into an argonaute (AGO) protein (generally AGO1). Thus MicroRNAs originate from primary transcripts (pri-miRNAs) containing hairpin structures.

In higher plants, miR156 regulates the vegetative phase change via the target SBP/SPL genes. The miR156 regulation during ontogenetic processes involves precursor genes of mature miR156.

In the context of the present invention, it is acknowledged that in plants, juvenile to adult phase transition is regulated by the sequential activity of two microRNAs (including precursor and mature miRNA): miR156 (also referred to as miRNA156) and miR172 (also referred to as miRNA172). A decline in miR156 and increase in miR172 abundance is associated with juvenile to adult phase transition. It is further within the scope of the present invention that miR156 binds and target transcripts of downstream genes, such as SQUAMOSA PROMOTER BINDING PROTEIN-LIKE (SPL) transcription factors (TFs) for degradation. miR156 is highly abundant in the juvenile phase and decreases as the plant ages, whereas miR172, a repressor of APETALA2 (AP2)-like TFs has the inverse transcript abundance. In general, as miR156 levels start declining, the level of its targeted downstream genes, such as SPL gene, start increasing or upregulating. The SPL genes, upregulate the transcription of miR172, resulting in AP2 TF repression, which marks the transition from juvenile to adult phase.

The microRNA 156 control of the vegetative phase change in plants and is under transcriptional regulation. miR156 represses flowering and plays an important role in the regulation of temperature-responsive flowering.

In general, vegetative phase changes in plants describes the transition between juvenile and adult phases of vegetative growth before flowering. It is one of the most fundamental mechanisms for plants to sense developmental signals, presenting a complex process involving many still-unknown determinants. Several studies in annual and perennial plants have identified the conservative roles of miR156 and its targets, SBP/SPL genes, in guiding the switch of plant growth from juvenile to adult phases. The current invention discloses methods to affect the regulatory mechanisms of miR156 regulation network, by downregulating miR156 using gene editing targeting miR156 sequence, preferably at the mature or conserved region. This will cause upregulation of miR156-regulated genes that are linked to plant developmental transition.

It is further noted that the transition of vegetative growth from the juvenile to the adult stage is essential for sexual reproduction. Many plants change vegetative traits with the development of their ability to flower, as differentiation pattern distinct between adult and juvenile organs. The current invention is aimed at downregulating miR156 by gene editing to upregulate downstream genes (e.g. SBP/SPL genes) that regulate adult vegetative differentiation to develop floral competence from the juvenile phase. This is based on the extremely conserved role of miR156 throughout the angiosperms, as evidenced from many species.

It is further acknowledged that miR156 accumulates at the highest level in the seedling stage, followed by a gradual decline with plant development, Plants that contain rich miR156 have a prolonged juvenile stage, which is characterized by increased branching, accelerated leaves emerging and retarded flowering. These changes that occur within the same plant are attributed to sequential alterations in miR156 and their squamosa promoter binding protein-like (SBP/SPL) targets.

The phrase “juvenile to adult phase transition” refers herein to the vegetative phase change (juvenile-to-adult vegetative transition). It further includes the floral transition, which refers to vegetative-to-reproductive transition. Thus the term “juvenile to adult phase transition” is herein used interchangeably with juvenile to reproductive phase transition. In general, the plant life cycle can be divided into three distinct growth phases—juvenile, adult, and reproductive—all of which involve temporal and spatially coordinated changes in various traits essential for plant survival and reproduction. These phases may be thought of as developmental phases, with continuous development of new organs that possess different morphological features in each phase. It is within the scope of the present invention that phase change transitions, associated with phenotypic changes, for example in leaves, are known to be moderated by the sequential action of two main microRNAs: miR156 and miR172.

By “modulates expression” it is herein meant an increase or a decrease in such expression.

As used herein the term “genetic modification” refers hereinafter to genetic manipulation or modulation, which is the direct manipulation of an organism's genes using biotechnology. It also refers to a set of technologies used to change the genetic makeup of cells, including the transfer of genes within and across species, targeted mutagenesis and genome editing technologies to produce improved organisms. According to main embodiments of the present invention, modified tree crop plants with improved traits, i.e. shorter juvenility time period, are generated using genome editing mechanism. This technique enables to achieve in planta modification of specific miRNAs/genes that relate to and/or control the juvenile to adult phase transition in tree crops.

The term “genome editing”, or “genome/genetic modification” or “genome engineering” generally refers hereinafter to a type of genetic engineering in which DNA is inserted, deleted, modified or replaced in the genome of a living organism. Unlike previous genetic engineering techniques that randomly insert genetic material into a host genome, genome editing targets the insertions to site specific locations. In the context of the present invention, the term also include base editing technique.

It is within the scope of the present invention that the common methods for such editing use engineered nucleases, or “molecular scissors”. These nucleases create site-specific double-strand breaks (DSBs) at desired locations in the genome. The induced double-strand breaks are repaired through nonhomologous end-joining (NHEJ) or homologous recombination (HR), resulting in targeted mutations (‘edits’). Families of engineered nucleases used by the current invention include, but are not limited to: meganucleases, zinc finger nucleases (ZFNs), transcription activator-like effector-based nucleases (TALEN), and the clustered regularly interspaced short palindromic repeats (CRISPR/Cas9) system.

The term “base editing” or “base-editing” in the context of the present invention refers to a genome editing approach that uses components from CRISPR systems together with other enzymes to directly introduce point mutations into cellular DNA or RNA without making double-stranded DNA breaks (DSBs). It is within the scope that DNA base editors comprise a catalytically disabled or inactivated nuclease (called herein dCas) fused to a nucleobase deaminase enzyme or a DNA glycosylase inhibitor. It is acknowledged that RNA base editors achieve analogous changes using components that target RNA. According to aspects of the present invention, base editors directly convert one base or base pair into another, enabling the efficient introduction of specific and precise point mutations in non-dividing cells without generating excess undesired editing byproducts such as indels, translocations, and rearrangements derived from DSBs created by nucleases such as Cas9 or any other Cas.

It is further within the scope of the current invention that DNA base editors (BEs) comprise fusions between a catalytically impaired Cas nuclease and a base-modification enzyme that operates on single-stranded DNA (ssDNA) but not double-stranded DNA (dsDNA). Without wishing to be bound by theory, it is noted that upon binding to its target locus in DNA, base pairing between the guide RNA and target DNA strand leads to displacement of a small segment of single-stranded DNA in an “R-loop”. DNA bases within this single-stranded DNA bubble are modified by the deaminase enzyme. To improve efficiency in eukaryotic cells, the catalytically disabled nuclease also generates a nick in the non-edited DNA strand, inducing cells to repair the non-edited strand using the edited strand as a template.

It is within the scope of the present invention that two classes of DNA base editor have been described: cytosine base editors (CBEs) which convert a C⋅G base pair into a T⋅A base pair, and adenine base editors (ABEs) which convert an A⋅T base pair to a G⋅C base pair. Together, CBEs and ABEs can mediate all four possible transition mutations (C to T, A to G, T to C, and G to A). In RNA, targeted adenosine conversion to inosine has been used in both antisense and Cas13-guided RNA-targeting methods.

Reference is now made to exemplary genome editing terms used by the current disclosure:

### Genome Editing Glossary

Cas = CRISPR-associated genes
Cas9, Csn1 = a CRISPR-associated protein
containing two nuclease domains, that is
programmed by small RNAs to cleave DNA
crRNA = CRISPR RNA
dCAS9 = nuclease-deficient Cas9
DSB = Double-Stranded Break
gRNA = guide RNA
HDR = Homology-Directed Repair
HNH = an endonuclease domain named
for characteristic histidine and asparagine
residues
Indel = insertion and/or deletion
NHEJ = Non-Homologous End Joining
PAM = Protospacer-Adjacent Motif
RuvC = an endonuclease domain named for
an E. coli protein involved in DNA repair
sgRNA = single guide RNA
tracrRNA, trRNA = trans-activating crRNA
TALEN = Transcription-Activator Like
Effector Nuclease
ZFN = Zinc-Finger Nuclease

According to specific aspects of the present invention, the CRISPR (Clustered Regularly Interspaced Short Palindromic Repeats) and CRISPR-associated (Cas) genes are used for the first time for generating genome modification in miRNAs (e.g. miRNA156) or their target genes in tree crops. It is herein acknowledged that the functions of CRISPR (Clustered Regularly Interspaced Short Palindromic Repeats) and CRISPR-associated (Cas) genes are essential in adaptive immunity in select bacteria and archaea, enabling the organisms to respond to and eliminate invading genetic material. These repeats were initially discovered in the 1980s in E. coli. Without wishing to be bound by theory, reference is now made to a type of CRISPR mechanism, in which invading DNA from viruses or plasmids is cut into small fragments and incorporated into a CRISPR locus comprising a series of short repeats (around 20 bps). The loci are transcribed, and transcripts are then processed to generate small RNAs (crRNA, namely CRISPR RNA), which are used to guide effector endonucleases that target invading DNA based on sequence complementarity.

According to further aspects of the invention, Cas protein, such as Cas9 (also known as Csn1) is required for gene silencing. Cas9 participates in the processing of crRNAs, and is responsible for the destruction of the target DNA. Cas9's function in both of these steps relies on the presence of two nuclease domains, a RuvC-like nuclease domain located at the amino terminus and a HNH-like nuclease domain that resides in the mid-region of the protein. To achieve site-specific DNA recognition and cleavage, Cas9 is complexed with both a crRNA and a separate trans-activating crRNA (tracrRNA or trRNA), that is partially complementary to the crRNA. The tracrRNA is required for crRNA maturation from a primary transcript encoding multiple pre-crRNAs. This occurs in the presence of RNase III and Cas9.

Without wishing to be bound by theory, it is herein acknowledged that during the destruction of target DNA, the HNH and RuvC-like nuclease domains cut both DNA strands, generating double-stranded breaks (DSBs) at sites defined by a 20-nucleotide target sequence within an associated crRNA transcript. The HNH domain cleaves the complementary strand, while the RuvC domain cleaves the noncomplementary strand.

It is further noted that the double-stranded endonuclease activity of Cas9 also requires that a short conserved sequence, (2-5 nts) known as protospacer-associated motif (PAM), follows immediately 3′—of the crRNA complementary sequence.

According to further aspects of the invention, a two-component system may be used by the current invention, combining trRNA and crRNA into a single synthetic single guide RNA (sgRNA) for guiding targeted gene alterations.

It is further within the scope that Cas9 nuclease variants include wild-type Cas9, Cas9D10A and nuclease-deficient Cas9 (dCas9).

Reference is now made to CRISPR/Cas9 mechanism of action as depicted by Xie, Kabin, and Yinong Yang. “RNA-guided genome editing in plants using a CRISPR-Cas system.” Molecular plant 6.6 (2013): 1975-1983. The Cas9 endonuclease forms a complex with a chimeric RNA (called guide RNA or gRNA), replacing the crRNA-transcrRNA heteroduplex, and the gRNA could be programmed to target specific sites. The gRNA-Cas9 should comprise at least 15-base-pairing (gRNA seed region) without mismatch between the 5′-end of engineered gRNA and targeted genomic site, and an NGG motif (called protospacer-adjacent motif or PAM) that follows the base-pairing region in the complementary strand of the targeted DNA.

It is within the scope of the present invention that the Cas gene may be selected from the group consisting of Cas3, Cas4, Cas5, Cas5e (or CasD), Cas6, Cas6e, Cas6f, Cas7, Cas8al, Cas8a2, Cas8b, Cas8c, Cas9, Cas10, Cast10d, Cas12, Cas13, Cas14, CasX, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, and Cu1966, bacteriophages Cas such as CasΦ (Cas-phi), split Cas such as split Cas12a or split Cas9 or nuclease-deactivated (dCas). and any combination thereof.

It is further within the scope of the present invention that the gRNA or sgRNA sequence comprises a 3′ Protospacer Adjacent Motif (PAM) selected from the group consisting of NGG (SpCas), NNNNGATT (NmeCas9), NNAGAAW (StCas9), NAAAAC (TdCas9), NNGRRT (SaCas9), TTTR PAM (archaea Cas12f) and TBN (Cas-phi).

A guide nucleic acid includes gRNA, gDNA, crRNA and crDNA.

The term “meganucleases” as used herein refers hereinafter to endodeoxyribonucleases characterized by a large recognition site (double-stranded DNA sequences of 12 to 40 base pairs); as a result this site generally occurs only once in any given genome. Meganucleases are therefore considered to be the most specific naturally occurring restriction enzymes.

The term “protospacer adjacent motif” or “PAM” as used herein refers hereinafter to a 2-6 base pair DNA sequence immediately following the DNA sequence targeted by the Cas9 nuclease in the CRISPR bacterial adaptive immune system. PAM is a component of the invading virus or plasmid, but is not a component of the bacterial CRISPR locus. PAM is an essential targeting component which distinguishes bacterial self from non-self DNA, thereby preventing the CRISPR locus from being targeted and destroyed by nuclease.

The term “plant-optimized nucleotide sequence” as used herein refers to a nucleotide sequence that has been optimized for increased expression in plants, particularly for increased expression in plants or in one or more plants of interest. For example, a plant-optimized nucleotide sequence can be synthesized by modifying a nucleotide sequence encoding a protein such as, for example, double-strand-break-inducing agent (e.g., an endonuclease) as disclosed herein, using one or more plant-preferred codons for improved expression.

The term “Next-generation sequencing” or “NGS” as used herein refers hereinafter to massively, parallel, high-throughput or deep sequencing technology platforms that perform sequencing of millions of small fragments of DNA in parallel. Bioinformatics analyses are used to piece together these fragments by mapping the individual reads to the reference genome.

The term “gene knockdown” as used herein refers hereinafter to an experimental technique by which the expression of one or more of an organism's genes is reduced. The reduction can occur through genetic modification, i.e. targeted genome editing or by treatment with a reagent such as a short DNA or RNA oligonucleotide that has a sequence complementary to either gene or an mRNA transcript. The reduced expression can be at the level of RNA or at the level of protein. It is within the scope of the present invention that the term gene knockdown also refers to a loss of function mutation and/or gene knockout mutation in which an organism's genes is made inoperative or nonfunctional.

The term “gene silencing” as used herein refers hereinafter to the regulation of gene expression in a cell to prevent the expression of a certain gene. Gene silencing can occur during either transcription or translation. In certain aspects of the invention, gene silencing is considered to have a similar meaning as gene knockdown. When genes are silenced, their expression is reduced. In contrast, when genes are knocked out, they are completely not expressed. Gene silencing may be considered a gene knockdown mechanism since the methods used to silence genes, such as RNAi, CRISPR, or siRNA, generally reduce the expression of a gene by at least 70% but do not completely eliminate it.

The term “loss of function mutation” as used herein refers to a type of mutation in which the altered gene product lacks the function of the wild-type gene. A synonyms of the term included within the scope of the present invention is null mutation.

The term “in planta” means in the context of the present invention within the plant or plant cells. More specifically, it means introducing CRISPR/Cas complex into plant material comprising a tissue culture of several cells, a whole plant, or into a single plant cell, without introducing a foreign gene or a mutated gene. It also used to describe conditions present in a non-laboratory environment (e.g. in vivo).

In some embodiments, the invention provides a nucleic acid sequence identical over its entire length to each genomic or RNA or coding sequence provided herein. The invention further provides a nucleic acid sequence displaying at least 80%, 85%, 90%, 95%, or 99% identity over its entire length to a the full length, or a fragment, of the genomic or RNA or coding sequence provided herein.

According to further embodiments, the nucleic acid can also include non-coding sequences, including for example, but not limited to, non-coding 5′ and 3′ sequences, such as miRNAs, the transcribed, untranslated sequences, termination signals, ribosome binding sites, sequences that stabilize mRNA, introns, polyadenylation signals, and additional coding sequence that encodes additional amino acids. Nucleic acids of the present invention also include nucleic acids comprising a structural gene or miRNA, and the naturally associated sequences that control gene expression or any target or downstream gene of the structural gene or miRNA.

The term ‘determinate’ or ‘determinate growth’ as used herein refers to plant growth in which the main stem ends in an inflorescence or other reproductive structure (e.g. a bud) and stops continuing to elongate indefinitely with only branches from the main stem having further and similarly restricted growth. It also refers to growth characterized by sequential flowering from the central or uppermost bud to the lateral or basal buds. It further means naturally self-limited growth, resulting in a plant of a definite maximum size.

The term ‘indeterminate’ or ‘indeterminate growth’ as used herein refers to plant growth in which the main stem continues to elongate indefinitely without being limited by a terminal inflorescence or other reproductive structure. It also refers to growth characterized by sequential flowering from the lateral or basal buds to the central or uppermost buds.

The term “orthologue” as used herein refers hereinafter to one of two or more homologous gene sequences found in different species.

The term “functional variant” or “functional variant of a nucleic acid or amino acid sequence” as used herein, refers to a variant of a sequence or part of a sequence which retains the biological function of the non-variant allele or sequence and hence has the activity of the expressed gene or protein or non-coding sequence. A functional variant also comprises a variant of the gene or non-coding sequence (e.g. miRNA) of interest which has sequence alterations that do not affect its function, for example, in non-conserved residues. Also encompassed is a variant that is substantially identical, i.e. has only some sequence variations, for example, in non-conserved residues, to the wild type nucleic acid or amino acid sequences as shown herein, and is biologically active.

In some aspects of the present invention, the term “variant” or “functional variant” as used throughout with reference to any of the sequences described herein refers to a variant gene sequence or part of the gene sequence (such as a fragment) or non-coding sequence (e.g. miRNA) which retains the biological function of the full non-variant sequence. A functional variant also comprises a variant of the sequence of interest, which has sequence alterations that do not affect function, for example in non-conserved residues. Also encompassed is a variant that is substantially identical, i.e. has only some sequence variations, for example in non-conserved residues, compared to the wild type sequences as shown herein and is biologically active. Each of the proposed modifications is well within the routine skill in the art, as is determination of retention of biological activity of the encoded products.

As used in any aspect of the invention described throughout a “variant” or a “functional variant” has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid or amino acid sequence.

According to some embodiments, a “functional variant” has at least 80% sequence identity to the non-variant nucleic acid or amino acid sequence.

The term “operably linked” as used throughout refers to a functional linkage between the promoter sequence and the gene or nucleotide sequence of interest, such that the promoter sequence is able to initiate transcription of the gene or nucleotide sequence of interest.

In a preferred embodiment of the invention, the regulatory sequence may be a promoter. According to aspects of the invention, including the method above and including the plants, methods and uses as described below, the term “regulatory sequence” is used interchangeably herein with “promoter” and all terms are to be taken in a broad context to refer to regulatory nucleic acid sequences capable of effecting expression of the sequences to which they are ligated. The term “regulatory sequence” also encompasses a synthetic fusion molecule or derivative that confers, activates or enhances expression of a nucleic acid molecule in a cell, tissue or organ.

The term “promoter” typically refers to a nucleic acid control sequence located upstream from the transcriptional start of a gene and which is involved in the binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a prokaryotic gene, in which case it may include a −35 box sequence and/or −10 box transcriptional regulatory sequences.

The term “variety” or “cultivar” used herein means a group of similar plants that by structural features and performance can be identified from other varieties within the same species.

The term “allele” used herein means any of one or more alternative or variant forms of a gene or a genetic unit at a particular locus, all of which alleles relate to one trait or characteristic at a specific locus. In a diploid cell of an organism, alleles of a given gene are located at a specific location, or locus (loci plural) on a chromosome. Alternative or variant forms of alleles may be the result of single nucleotide polymorphisms, insertions, inversions, translocations or deletions, or the consequence of gene regulation caused by, for example, by chemical or structural modification, transcription regulation or post-translational modification/regulation. An allele associated with a qualitative trait may comprise alternative or variant forms of various genetic units including those mat are identical or associated with a single gene or multiple genes or their products or even a gene disrupting or controlled by a genetic factor contributing to the phenotype represented by the locus. According to further embodiments, the term “allele” designates any of one or more alternative forms of a gene at a particular locus. Heterozygous alleles are two different alleles at the same locus. Homozygous alleles are two identical alleles at a particular locus. A wild type allele is a naturally occurring allele.

As used herein, the term “locus” (loci plural) means a specific place or places or region or a site on a chromosome where for example a gene or genetic marker element or factor is found. In specific embodiments, such a genetic element is contributing to a trait.

As used herein, the term “homozygous” refers to a genetic condition or configuration existing when two identical or like alleles reside at a specific locus, but are positioned individually on corresponding pairs of homologous chromosomes in the cell of a diploid organism.

Conversely, as used herein, the term “heterozygous” means a genetic condition or configuration existing when two different or unlike alleles reside at a specific locus, but are positioned individually on corresponding pairs of homologous chromosomes in the cell of a diploid organism.

As used herein, the phrase “genetic marker” or “molecular marker” or “biomarker” refers to a feature in an individual's genome e.g., a nucleotide or a polynucleotide sequence that is associated with one or more loci or trait of interest In some embodiments, a genetic marker is polymorphic in a population of interest, or the locus occupied by the polymorphism, depending on context. Genetic markers or molecular markers include, for example, single nucleotide polymorphisms (SNPs), indels (i.e. insertions deletions), simple sequence repeats (SSRs), restriction fragment length polymorphisms (RFLPs), random amplified polymorphic DNAs (RAFDs), cleaved amplified polymorphic sequence (CAPS) markers, Diversity Arrays Technology (DArT) markers, and amplified fragment length polymorphisms (AFLPs) or combinations thereof, among many other examples such as the DNA sequence per se. Genetic markers can, for example, be used to locate genetic loci containing alleles on a chromosome that contribute to variability of phenotypic traits. The phrase “genetic marker” or “molecular marker” or “biomarker” can also refer to a polynucleotide sequence complementary or corresponding to a genomic sequence, such as a sequence of a nucleic acid used as a probe or primer.

As used herein, the term “germplasm” refers to the totality of the genotypes of a population or other group of individuals (e.g., a species). The term “germplasm” can also refer to plant material; e.g., a group of plants that act as a repository for various alleles. Such germplasm genotypes or populations include plant materials of proven genetic superiority; e.g., for a given environment or geographical area, and plant materials of unknown or unproven genetic value; that are not part of an established breeding population and that do not have a known relationship to a member of the established breeding population.

The terms “hybrid”, “hybrid plant” and “hybrid progeny” used herein refers to an individual produced from genetically different parents (e.g., a genetically heterozygous or mostly heterozygous individual).

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins, it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. The term further refers hereinafter to the amount of characters which match exactly between two different sequences. Hereby, gaps are not counted and the measurement is relational to the shorter of the two sequences.

The term “identity” as used herein and well understood in the art, refers to a relationship between two or more polypeptide sequences or two or more polynucleotide sequences, as determined by comparing the sequences. It also means the degree of sequence relatedness between polypeptide or polynucleotide sequences, as determined by the match between strings of such sequences. Methods to determine “identity” are designed to give the largest match between the sequences tested. “Identity” can be readily calculated or determined by known methods. Computer programs can be used to compare or determine “identity” between two sequences these programs include but are not limited to, GCG; suite of BLAST programs, three designed for nucleotide sequences queries (BLASTN, BLASTX, and TBLASTX) and two designed for protein sequence queries (BLASTP and TBLASTN). The BLASTX program is publicly available from NCBI and other sources.

It is further within the scope that the terms “similarity” and “identity” additionally refer to local homology, identifying domains that are homologous or similar (in nucleotide and/or amino acid sequence). It is acknowledged that bioinformatics tools such as BLAST, SSEARCH, FASTA, and HMMER calculate sequence comparison or alignments which identify similar position or region between two sequences. For domains that are found in different sequence contexts in different proteins, the alignment should be limited to the homologous domain, since the domain homology is providing the sequence similarity captured in the score. According to some aspects the term similarity or identity further includes a sequence motif, which is a nucleotide or amino-acid sequence pattern that is widespread and has, or is conjectured to have, a biological significance. Proteins may have a sequence motif and/or a structural motif, a motif formed by the three-dimensional arrangement of amino acids which may not be adjacent.

As used herein, the terms “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene”, “allele” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences. Thus, according to the various aspects of the invention, genomic DNA, cDNA or coding DNA may be used. In one embodiment, the nucleic acid is cDNA or coding DNA.

The terms “peptide”, “polypeptide” and “protein” are used interchangeably herein and refer to amino acids in a polymeric form of any length, linked together by peptide bonds.

According to other aspects of the invention, a “modified” or a “mutant” plant is a plant that has been altered compared to the naturally occurring wild type (WT) plant.

As used herein, a “desirable trait” or “desirable traits” include, but are not limited to: reduced or shorter juvenility phase period, early juvenile to adult phase transition, increased vegetative and/or reproductive growth, improved vegetative and/or reproductive or fruit/vegetable yield, and improved biomass yield. It is further within the scope that the tree crops of the present invention are characterized by enhanced productivity trait (fruit or vegetable yield), enhanced agronomic trait and/or enhanced plant biomass.

According to some core embodiments, the tree crop produced by the method of the present invention exhibits time reduced juvenility or vegetative phase change, increased duration of adult or mature phase, and/or early transition into adult or mature or reproductive phase, relative to a corresponding tree plant lacking the targeted genomic modification in an endogenous microRNA156 (miRNA156), or a downstream gene target of the miRNA156.

As used herein, “polymorphism” means the presence of one or more variations of a nucleic acid sequence at one or more loci in a population of one or more individuals. The variation may comprise but is not limited to one or more base changes, the insertion of one or more nucleotides or the deletion of one or more nucleotides, A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found, or may exist at low frequency within a population, the former having greater utility in general plant breeding and the latter may be associated with rare but important phenotypic variation. Common polymorphisms may include single nucleotide polymorphisms (SNPs), insertions or deletions in DNA sequence (Indels), simple sequence repeats of DNA sequence (SSRs) a restriction fragment length polymorphism, and a tag SNP. A genetic marker, a gene, a DNA-derived sequence, a haplotype, a RNA-derived sequence, a promoter, a 5′ untranslated region of a gene, a 3′ untranslated region of a gene, microRNA, siRNA, a QTL, a satellite marker, a transgene, mRNA, dsRNA, a transcriptional profile, and a methylation pattern may comprise polymorphisms. In addition, the presence, absence, or variation in copy number of the preceding may comprise a polymorphism.

As used herein, “genotype” is the actual nucleic acid sequence at a locus in an individual plant. As used herein, “phenotype” means the detectable characteristics (e.g. number of juvenile leaves, or timing of production of leaves displaying adult morphological characteristics, such as the presence of waxes) of a cell or organism which can be influenced by genotype.

Main aspects of the invention involve targeted mutagenesis methods, specifically genome editing, and exclude embodiments that are solely based on generating plants by traditional breeding methods. In a further embodiment of the current invention, as explained herein, the improved domestication at least one trait is not due to the presence of a transgene.

The loss of function mutation may be a deletion or insertion (“indels”) with reference the wild type or endogenous microRNA156 (miRNA156), or a downstream gene target of the miRNA156. The deletion may comprise 1-20 or more nucleotides, for example 1, 2, 3, 4, 5, 6, 7, 8, 9, 10, 1, 12, 13, 14, 15, 16, 17, 18 or 20 nucleotides or more in one or more strand. The insertion may comprise 1-20 or more nucleotides, for example 1, 2, 3, 4, 5, 6, 7, 8, 9, 10, 1, 12, 13, 14, 15, 16, 17, 18 or 20 or more nucleotides in one or more strand.

The plant of the invention includes plants wherein the plant is heterozygous for the each of the mutations. In a preferred embodiment however, the plant is homozygous for the mutations. Progeny that is also homozygous can be generated from these plants according to methods known in the art.

It is further within the scope that variants of a particular microRNA, or a downstream gene target of the miRNA nucleotide sequence according to the various aspects of the invention will have at least about 50%-99%, for example at least 75%, for example at least 85%, 86%, 87%, 88%, 89%, 90%, 92%, 94%, 95%, 96%, 97%, 98% or 99% or more sequence identity to that particular nucleotide sequence. Sequence alignment programs to determine sequence identity are well known in the art.

Also, the various aspects of the invention encompass not only a microRNA156 (miRNA156), or a downstream gene target of the miRNA156 nucleic acid sequence, but also fragments thereof. By “fragment” is intended a portion of the nucleotide sequence or a portion of the amino acid sequence and hence of the protein encoded thereby. Fragments of a nucleotide sequence may encode protein fragments that retain the biological activity of the native protein, in this case improved domestication trait.

According to further embodiments of the present invention, DNA introduction into the plant cells can be done by Agrobacterium infiltration, virus based plasmids for delivery of the genome editing molecules and mechanical insertion of DNA (PEG mediated DNA transformation, biolistics, etc.).

It is further within the scope that suitable methods for transformation of plant or cells thereof for use with the current invention may include any method by which DNA can be introduced into a cell, such as by direct delivery of DNA such as by PEG-mediated transformation of protoplasts. These methods and their use are well known in the art.

After effecting delivery of exogenous DNA to recipient cells, the next steps generally concern identifying the transformed cells for further culturing and plant regeneration. In order to improve the ability to identify transformants, one may employ a selectable or screenable marker gene with a transformation vector prepared in accordance with the invention. In this case, one would then generally assay the potentially transformed cell population by exposing the cells to a selective agent or agents, or one would screen the cells for the desired marker gene trait.

Cells that survive the exposure to the selective agent, or cells that have been scored positive in a screening assay, may be cultured in media that supports regeneration of plants.

To confirm the presence of the exogenous DNA or “transgene(s)” in the regenerating plants, a variety of assays may be performed. Such assays include, for example, “molecular biological” assays, such as Southern and northern blotting and PCR; “biochemical” assays, such as detecting the presence of a protein product, e.g., by immunological means (ELISAs and western blots) or by enzymatic function; plant part assays, such as leaf or root assays; and also, by analyzing the phenotype of the whole regenerated plant. These phenotypic assays may include in a non-limiting manner, analyzing changes in the chemical composition, morphology, or physiological and/or developmental properties of the plant. Morphological changes may include ones known to demonstrate juvenile or adult characteristics in plant vegetative or reproductive tissues, such as early flowering and early fruit setting compared to wild type non edited corresponding trees.

The present invention provides a seed of a plant, especially a tree plant, capable of producing a plant having reduced juvenile growth phase and/or enhanced adult growth phase. In one aspect, the plant can be an open-pollinated variety, a hybrid parent inbred line, or a male sterile line.

In another aspect, tissue culture of the plants described herein relates to the culture of protoplasts, calli, or plant cells or regenerated plant cells that are isolated from, or present in, intact parts of the plants described herein.

Once the tree plants are produced which display an enhanced, e.g. extended, adult phase of growth and reduced juvenile growth phase, the tree plants themselves can be cultivated in accordance with conventional procedures, including via tissue culture and by sexual reproduction. The seeds resulting from the tree plants can be recovered and planted or otherwise grown as a means of propagation. Plants may also be obtained through asexual reproduction. Protoplast or propagules (e.g., cuttings, scions or rootstocks) can be recovered from plants or parts thereof and may be employed to propagate additional plants.

The present invention also provides and includes a container of seeds characterized by extended adult phase and reduced duration of juvenile phase.

One aspect of the invention relates to vegetative tissues, including tissues harvested, dried, or otherwise processed, biomass produced by a tree plant having a genome that comprises at least one genome editing modification giving rise to an enhanced adult phase of growth.

The present invention also provides progeny of plants displaying extended adult growth and reduced juvenility phase of growth. As used herein, progeny include not only, without limitation, the products of any cross (be it a backcross or otherwise) between two plants, but all progeny whose pedigree traces back to the original cross.

In addition, it is within the scope of the present invention that the Cas9 protein is directly inserted together with a gRNA (ribonucleoprotein—RNP's) in order to bypass the need for in vivo transcription and translation of the Cas9+gRNA plasmid in planta to achieve gene editing.

According to a further embodiment of the present invention, the gene modification is introduced using CRISPR (Clustered Regularly Interspaced Short Palindromic Repeats) and CRISPR-associated (Cas) gene (CRISPR/Cas) system, Transcription activator-like effector nuclease (TALEN), Zinc Finger Nuclease (ZFN), meganuclease or any combination thereof.

According to a further embodiment of the present invention, the targeted gene modification is introduced using (i) at least one RNA guided endonuclease, or a nucleic acid encoding at least one RNA-guided endonuclease, and (ii) at least one guide RNA (gRNA) or DNA encoding at least one gRNA which directs the endonuclease to a corresponding target sequence within microRNA156 (miRNA156). According to a further embodiment upregulating of a downstream gene target of the miRNA156 is performed in the tree plant (e.g. by gene editing targeted to silencing miRNA156).

It is also possible to create a genome edited plant and use it as a rootstock. Then, the Cas protein and gRNA can be transported via the vasculature system to the top of the plant and create the genome editing event in the scion.

It is within the scope of the present invention that the usage of CRISPR/Cas system for the generation of tree plants with earlier juvenile to adult phase transition by introducing targeted at least one genomic modification silencing the expression of an endogenous microRNA156 (miRNA156), or a downstream gene target of the miRNA156, allows the modification of predetermined specific DNA sequences without introducing foreign DNA into the genome by GMO techniques. According to one embodiment of the present invention, this is achieved by combining the Cas nuclease (e.g. Cas9, Cpf1 and the like) with a predefined guide RNA molecule (gRNA). The gRNA is complementary to a specific DNA sequence targeted for editing in the plant genome and which guides the Cas nuclease to a specific nucleotide sequence. The predefined gene specific gRNA's are cloned into the same plasmid as the Cas gene and this plasmid is inserted into plant cells. Insertion of the aforementioned plasmid DNA can be done, but not limited to, using different delivery systems, biological and/or mechanical, e.g. Agrobacterium infiltration, virus based plasmids for delivery of the genome editing molecules and mechanical insertion of DNA (PEG mediated DNA transformation, biolistics, etc.).

It is further within the scope of the present invention that upon reaching the specific predetermined DNA sequence, the Cas9 nuclease cleaves both DNA strands to create double stranded breaks leaving blunt ends. This cleavage site is then repaired by the cellular non homologous end joining DNA repair mechanism resulting in insertions or deletions which eventually create a mutation at the cleavage site. For example, it is acknowledged that a deletion form of the mutation consists of at least 1 base pair deletion. As a result of this base pair deletion the gene coding sequence is disrupted and the translation of the encoded protein is compromised either by a premature stop codon or disruption of a functional or structural property of the protein. Thus DNA is cut by the Cas9 protein and re-assembled by the cell's DNA repair mechanism.

In order to understand the invention and to see how it may be implemented in practice, a plurality of preferred embodiments will now be described, by way of non-limiting example only, with reference to the following examples.

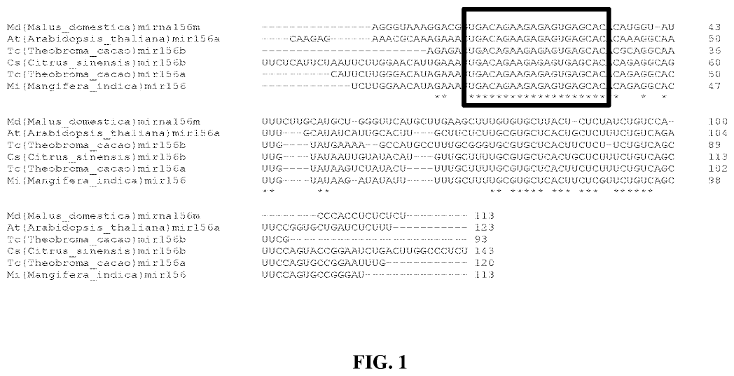

Reference is now made to FIG. 1 presenting a sequence comparison of microRNA156 (miR156) precursors from various representative tree crop species. The presented box highlights the mature miRNA sequence which is the actual sequence that bind the mRNA targeted for degradation. Through this sequence gene expression is regulated and it is highly conserved in all mentioned tree crops.

This figure shows that there are conserved regions between miR156 variants or homologs from different tree crop species (e.g. Malus domestica, Arabidopsis thaliana, Theobroma cacao, Citrus x sinensis and Mangifera indica). This means that miR156 homologs share conserved specific regions amongst various tree crop species. The highlighted conserved region forms the mature miR156, which binds the target genes and regulate their expression.

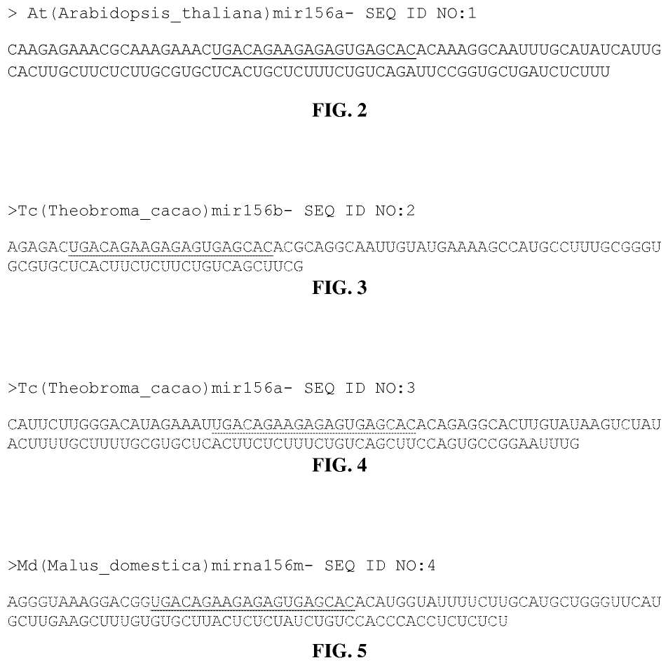

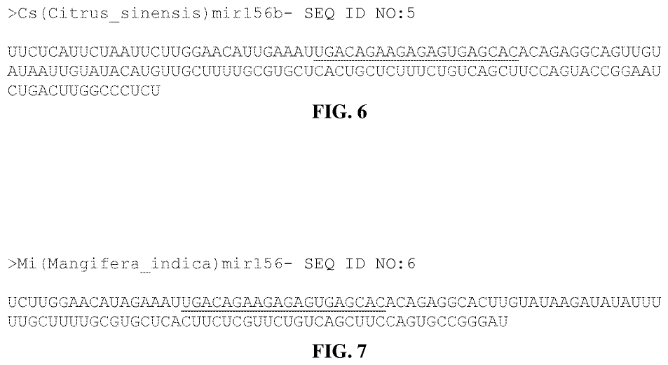

Reference is now made to FIGS. 2-7 presenting: Arabidopsis thaliana miR156a (At miR156a) sequence as set forth in SEQ ID NO:1, Theobroma cacao miR156b (Tc miR156b) sequence as set forth in SEQ ID NO:2, Theobroma cacao miR156a (Tc miR156a) sequence as set forth in SEQ ID NO:3, Malus domestica miR156m (Md miR156m) sequence as set forth in SEQ ID NO:4, Citrus x sinensis miR156b (Cs miR156b) sequence as set forth in SEQ ID NO:5, and Mangifera indica miR156 (Mi miR156) sequence as set forth in SEQ ID NO:6, respectively. These figures show that all presented miR156 sequences comprise a region of 20 nucleotides, which forms the mature miR156 and is conserved amongst the different tree crop species. This implies the importance of this region in regulating target genes such as SQUAMOSA promoter binding protein-like (SPL or SBP box) genes. By the current invention, this region can be targeted by gene editing to modulate juvenile to adult phase transition (shorter the juvenility phase period) in different crop trees, to enhance yield.

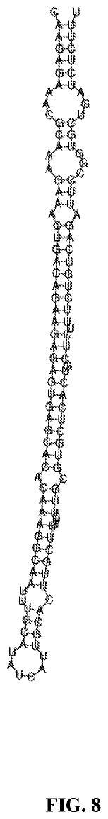

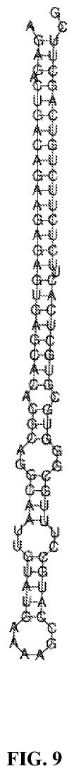

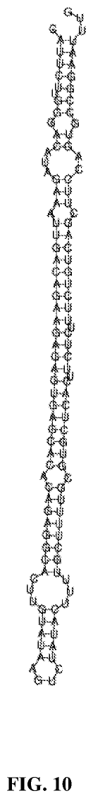

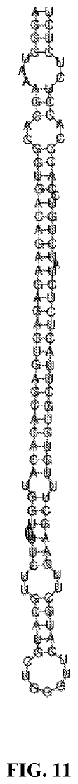

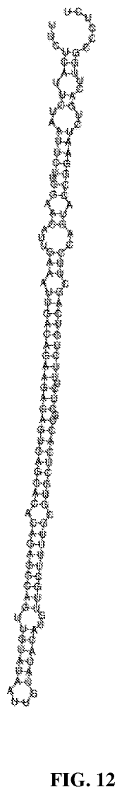

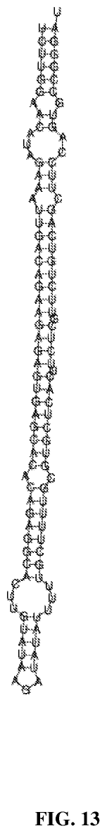

FIGS. 8-13 present the secondary structural prediction of At miR156a, Tc miR156b, Tc miR156a, Md miR156m, Cs miR156b, and Mi miR156 sequences, respectively, using RNAfold web server program (rna.tbi.univie.ac.at//cgi-bin/RNAWebSuite/RNAfold.cgi). It can be seen that in the various selected tree crops, miR156 precursors present a similar secondary structure. The different miR156 sequences are conserved mainly within the hairpin region. Thus miR156 from different tree species share sequence and secondary structure similarity.

This shows that by using the gene editing method described by the present invention, encompassing the CRISPR/Cas technique, silencing of miR156 regulation activity can be achieved by targeted editing of conserved regions amongst miR156 in various tree crop species. In this way the desirable trait of shorter juvenility phase period or early juvenile to adult phase transition, can be generated in valuable tree crops to increase their yield.

According to one embodiment, the present invention provides a method for controlling juvenile to adult phase transition in a tree plant, the method comprising introducing using targeted genome editing, at least one genomic modification in an endogenous microRNA156 (miRNA156).

According to a further embodiment, the present invention provides the method as defined above, wherein the method comprises upregulation of a downstream gene target of the miRNA156.

According to a further embodiment, the present invention provides the method as defined in any of the above, wherein the method comprises expressing in the tree plant a nuclease comprising a nucleic acid binding domain, wherein the nucleic acid binding domain binds to a target site in the miRNA156, to generate the genomic modification.

According to a further embodiment, the present invention provides the method as defined in any of the above, wherein the nuclease is selected from CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease, or any combination thereof.

According to a further embodiment, the present invention provides the method as defined in any of the above, wherein the nuclease introduces a mutation in the miRNA156 via a DNA cleavage domain that cleaves the miRNA156 encoding gene, or via base editing of the miRNA156 encoding gene.

According to a further embodiment, the present invention provides the method as defined in any of the above, wherein the method comprises expressing in the tree plant a guide RNA nucleic acid that binds to a target site in the miRNA156, and a CRISPR-Cas endonuclease that associates with the gRNA nucleic acid.

According to a further embodiment, the present invention provides the method as defined in any of the above, wherein said genomic modification is generated via introduction of (a) Cas DNA or a nuclease-deactivated (dCas) and gRNA polynucleotide comprising a nucleic acid sequence complementary to a target site in the miRNA156, or (b) a ribonucleoprotein (RNP) complex comprising Cas protein and gRNA polynucleotide comprising a nucleic acid sequence complementary to a target site in the miRNA156, and any combination thereof.

According to a further embodiment, the present invention provides the method as defined in any of the above, wherein said gRNA sequence comprises a 3′ Protospacer Adjacent Motif (PAM) selected from the group consisting of NGG (SpCas), NNNNGATT (NmeCas9), NNAGAAW (StCas9), NAAAAC (TdCas9), NNGRRT (SaCas9), TTTR PAM (archaea Cas12f), and TBN (Cas-phi).

According to a further embodiment, the present invention provides the method as defined in any of the above, wherein said Cas gene is selected from the group consisting of Cas3, Cas4, Cas5, Cas5e (or CasD), Cas6, Cas6e, Cas6f, Cas7, Cas8al, Cas8a2, Cas8b, Cas8c, Cas9, Cas10, Cast10d, Cas12, Cas13, Cas14, CasX, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, and Cu1966, bacteriophages Cas such as CasΦ (Cas-phi), split Cas such as split Cas12a or split Cas9 or nuclease-deactivated (dCas) and any combination thereof.

According to a further embodiment, the present invention provides the method as defined in any of the above, wherein said tree plant comprises a DNA construct, said DNA construct comprising a promoter operably linked to a nucleotide sequence encoding a plant optimized Cas endonuclease, said plant optimized Cas endonuclease is capable of binding to and creating a double strand break in a genomic target sequence of said plant genome.

According to a further embodiment, the present invention provides the method as defined in any of the above, wherein said genomic modification is a silencing mutation, a knockdown mutation, a knockout mutation, a loss of function mutation or any combination thereof.

According to a further embodiment, the present invention provides the method as defined in any of the above, wherein said genomic modification is an insertion, deletion, indel or a substitution.

According to a further embodiment, the present invention provides the method as defined in any of the above, wherein said genomic modification is an induced mutation in the coding region of said gene, in a conserved region within the gene, a mutation in the regulatory region of said gene, a mutation in a gene downstream or upstream of miRNA156, and/or an epigenetic factor.

According to a further embodiment, the present invention provides the method as defined in any of the above, wherein said method comprises reducing the duration of juvenile phase in the tree plant by introduction of a targeted genomic modification in the microRNA156 (miRNA156), thereby down regulating expression of the endogenous microRNA156 (miRNA156) and/or upregulating of a downstream gene target of the miRNA156.

According to a further embodiment, the present invention provides the method as defined in any of the above, wherein the downstream gene target of the miRNA156 is selected from genes comprising a miR156 recognition site or element.

According to a further embodiment, the present invention provides the method as defined in any of the above, wherein the downstream gene target of the miRNA156 is selected from genes encoding SQUAMOSA PROMOTER BINDING PROTEIN (SBP)-domain containing transcription factors, SQUAMOSA PROMOTER BINDING PROTEIN-LIKE (SPL) transcription factors and SUPPRESSOR OF OVEREXPRESSION OF CONSTANS 1 (SOC1).

According to a further embodiment, the present invention provides the method as defined in any of the above, wherein the tree plant is selected from horticultural tree crops such as fruit tree species including: Theobroma cacao (cacao), Mangifera indica (mango), Persea Americana (avocado), Macadamia integrifolia (macadamia), Prunus dulcis (almond), Malus domestica (apple), Prunus armeniaca (apricot), Averrhoa carambola (carambola, starfruit), Anacardium occidentale (cashew), Prunus avium (cherry), citrus (orange, lemon, lime, etc.), Cocos nucifera (coconut), Citrus x paradisi (grapefruit), Psidium guajava (guava), Litchi chinensis (lychee), Olea europaea (olive), Prunus persica (peach) and Prunus persica var. nucipersica (nectarine), Pyrus (pear), Carya illinoinensis (pecan), Diospyros kaki (persimmon), Prunus domestica (plum), Citrus maxima (pomelo), Punica granatum (pomegranate), Castanea sativa (sweet chestnut), Solanum betaceum (tamarillo) and Juglans (walnut).

According to a further embodiment, the present invention provides the method as defined in any of the above, wherein the tree plant comprises enhanced productivity trait (fruit or vegetable yield), enhanced agronomic trait and/or enhanced plant biomass.

According to a further embodiment, the present invention provides the method as defined in any of the above, wherein the tree crop exhibits time reduced juvenility or vegetative phase change, increased duration of adult or mature phase, and/or early transition into adult or mature or reproductive phase, relative to a corresponding tree plant lacking the targeted genomic modification.

According to a further embodiment, the present invention provides the method as defined in any of the above, wherein the method comprises modulating the timing of juvenile to adult phase transition, the timing of juvenile to adult phase transition is calculated based on miRNA156 target genes expression level and is shorter compared to wild type non edited corresponding plants.

According to a further embodiment, the present invention provides the method as defined in any of the above, wherein the tree plant exhibits an altered trait selected from the group consisting of: an altered proportion of juvenile, transitional, or adult leaves; enhanced yield of vegetative tissue; enhanced fruit yield, altered development or morphology, wherein the trait exhibited by the tree plant is altered relative to a corresponding wild type tree plant.

It is further within the scope to provide a method for controlling juvenile to adult phase transition in a tree plant, the method comprising using targeted genome editing, generating at least one genomic modification in an endogenous microRNA156 (miRNA156) and/or upregulating a downstream gene target of the miRNA156.

It is further within the scope to provide a tree plant, plant cell, plant tissue, plant part or a seed, produced by the method as defined in any of the above.

It is further within the scope of the present invention to disclose a tree plant exhibiting modulated timing of juvenile to adult phase transition, the tree plant comprises introduced targeted genome modification in a microRNA156 (miRNA156), and/or upregulated expression of a downstream gene target of the miRNA156.

It is further within the scope of the present invention to disclose the tree plant as defined in any of the above, wherein said tree plant exhibits time reduced juvenility or vegetative phase change, increased duration of adult or mature phase, and/or early transition into adult or mature or reproductive phase, relative to a corresponding tree plant lacking the targeted genomic modification.

It is further within the scope of the present invention to disclose the tree plant as defined in any of the above, wherein the tree plant is expressing a nuclease comprising a nucleic acid binding domain, wherein the nucleic acid binding domain binds to a target site in the miRNA156.

It is further within the scope of the present invention to disclose the tree plant as defined in any of the above, wherein the nuclease is selected from CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease, or any combination thereof.

It is further within the scope of the present invention to disclose the tree plant as defined in any of the above, wherein the nuclease introduces a mutation in the miRNA156 via a DNA cleavage domain that cleaves the miRNA156, or via base editing of the miRNA156.

It is further within the scope of the present invention to disclose the tree plant as defined in any of the above, wherein the tree plant is expressing a guide RNA nucleic acid that binds to a target site in the miRNA156, and a CRISPR-Cas endonuclease that associates with the gRNA nucleic acid.

It is further within the scope of the present invention to disclose the tree plant as defined in any of the above, wherein said genomic modification is generated via introduction of (a) Cas DNA and gRNA polynucleotide comprising a nucleic acid sequence complementary to a target site in the miRNA156, or (b) a ribonucleoprotein (RNP) complex comprising Cas and gRNA polynucleotide comprising a nucleic acid sequence complementary to a target site in the miRNA156, and any combination thereof.

It is further within the scope of the present invention to disclose the tree plant as defined in any of the above, wherein said gRNA sequence comprises a 3′ Protospacer Adjacent Motif (PAM) selected from the group consisting of NGG (SpCas), NNNNGATT (NmeCas9), NNAGAAW (StCas9), NAAAAC (TdCas9), NNGRRT (SaCas9), TTTR PAM (archaea Cas12f), and TBN (Cas-phi).

It is further within the scope of the present invention to disclose the tree plant as defined in any of the above, wherein said Cas gene is selected from the group consisting of Cas3, Cas4, Cas5, Cas5e (or CasD), Cas6, Cas6e, Cas6f, Cas7, Cas8a1, Cas8a2, Cas8b, Cas8c, Cas9, Cas10, Cast10d, Cas12, Cas13, Cas14, CasX, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, and Cu1966, bacteriophages Cas such as CasΦ (Cas-phi), split Cas such as split Cas12a or split Cas9 or nuclease-deactivated (dCas) and any combination thereof.

It is further within the scope of the present invention to disclose the tree plant as defined in any of the above, wherein said tree plant comprises a DNA construct, said DNA construct comprising a promoter operably linked to a nucleotide sequence encoding a plant optimized Cas endonuclease, said plant optimized Cas endonuclease is capable of binding to and creating a double strand break in a genomic target sequence of said plant genome.

It is further within the scope of the present invention to disclose the tree plant as defined in any of the above, wherein said genomic modification is a silencing mutation, a knockdown mutation, a knockout mutation, a loss of function mutation or any combination thereof.

It is further within the scope of the present invention to disclose the tree plant as defined in any of the above, wherein said genomic modification is an insertion, deletion, indel or a substitution.

It is further within the scope of the present invention to disclose the tree plant as defined in any of the above, wherein said genomic modification is an induced mutation in the coding region of said gene, in a conserved region within the gene, a mutation in the regulatory region of said gene, a mutation in a gene downstream or upstream of miRNA156, and/or an epigenetic factor.

It is further within the scope of the present invention to disclose the tree plant as defined in any of the above, wherein said tree plant exhibits reduced duration of the juvenile phase, further wherein the plant comprises a targeted genomic modification in the microRNA156 (miRNA156), said modification down regulate expression of the endogenous microRNA156 (miRNA156) and/or upregulate expression of a downstream gene target of the miRNA156.

It is further within the scope of the present invention to disclose the tree plant as defined in any of the above, wherein the downstream gene target of the miRNA156 is selected from genes comprising a miR156 recognition site or element.

It is further within the scope of the present invention to disclose the tree plant as defined in any of the above, wherein the downstream gene target of the miRNA156 is selected from genes encoding SQUAMOSA PROMOTER BINDING PROTEIN (SBP)-domain containing transcription factors, SQUAMOSA PROMOTER BINDING PROTEIN-LIKE (SPL) transcription factors and SUPPRESSOR OF OVEREXPRESSION OF CONSTANS 1 (SOC1).

It is further within the scope of the present invention to disclose the tree plant as defined in any of the above, wherein the tree plant is selected from horticultural tree crops such as fruit tree species including: Theobroma cacao (cacao), Mangifera indica (mango), Persea Americana (avocado), Macadamia integrifolia (macadamia), Prunus dulcis (almond), Malus domestica (apple), Prunus armeniaca (apricot), Averrhoa carambola (carambola, starfruit), Anacardium occidentale (cashew), Prunus avium (cherry), citrus (orange, lemon, lime, etc.), Cocos nucifera (coconut), Citrus x paradisi (grapefruit), Psidium guajava (guava), Litchi chinensis (lychee), Olea europaea (olive), Prunus persica (peach) and Prunus persica var. nucipersica (nectarine), Pyrus (pear), Carya illinoinensis (pecan), Diospyros kaki (persimmon), Prunus domestica (plum), Citrus maxima (pomelo), Punica granatum (pomegranate), Castanea sativa (sweet chestnut), Solanum betaceum (tamarillo) and Juglans (walnut).

It is further within the scope of the present invention to disclose the tree plant as defined in any of the above, wherein the tree plant comprises enhanced productivity trait (fruit or vegetable yield), enhanced agronomic trait and/or enhanced plant biomass.

It is further within the scope of the present invention to disclose the tree plant as defined in any of the above, wherein the timing of juvenile to adult phase transition is shorter compared to wild type non edited corresponding plants.

It is further within the scope of the present invention to disclose the tree plant as defined in any of the above, wherein the tree plant exhibits an altered trait selected from the group consisting of: an altered proportion of juvenile, transitional, or adult leaves; enhanced yield of vegetative tissue; enhanced fruit yield, altered development or morphology, wherein the trait exhibited by the tree plant is altered relative to a corresponding wild type tree plant.

It is further within the scope of the present invention to disclose a plant cell, plant tissue, regenerable cells, plant part or a seed of the tree plant as defined in any of the above.

It is further within the scope of the present invention to disclose a polynucleotide molecule comprising a promoter operably linked to a nucleotide sequence encoding a Cas nuclease and a guide RNA (gRNA) comprising a nucleic acid sequence complementary to a target site in miRNA156

It is further within the scope of the present invention to disclose a recombinant vector comprising the polynucleotide molecule as defined in any of the above.

It is further within the scope of the present invention to disclose the recombinant vector as defined in any of the above, further comprising at least one additional sequence selected from: a regulatory sequence, a selectable marker, a leader sequence and a terminator.

### Example 1

Production of Tree Crops with Modulated, Preferably Early, Timing of Juvenile to Adult Phase Transition by Targeted Genome Editing

This example describes a generalized scheme of the process for generating the genome edited tree plants of the present invention. The process comprises the following steps:
1. Designing and synthesizing gRNA's corresponding to a sequence targeted for editing. Editing event should be designed flanking with a unique restriction site sequence to allow easier screening of successful editing.
2. Carrying transformation using Agrobacterium or biolistics. For Agrobacterium and biolistics transformation using a DNA plasmid, a vector containing a selection marker, Cas9 gene and relevant gRNA's is constructed. For biolistics using Ribonucleoprotein (RNP) complexes, RNP complexes are created by mixing the Cas9 protein with relevant gRNA's.
3. Performing regeneration in tissue culture. For DNA transformation, using antibiotics for selection of positive transformants.
4. Selecting positive transformants. Once regenerated plants appear in the regenerated tissue culture, obtaining leaf (or any other selected tissue) samples, extracting DNA from the obtained sample and preforming PCR using primers flanking the editing region. The resulted PCR products are digested with enzymes recognizing the restriction site near original gRNA sequence. If editing event occurred, the restriction site will be disrupted and PCR product will not be cleaved. Absence of an editing event will result in a cleaved PCR product.

It is within the scope of the current invention that genetic markers specific to the above mentioned editing events and/or phenotypes, for various tree crops are developed and provided by the current invention.

It is further within the scope of the current invention that allele and genetic variation is analyzed for the tree crop strains used.

Reference is now made to optional stages that have been used for the production of mutated miRNA156, or upregulation of downstream gene targets of the miRNA156, by genome editing:
Stage 1: Identifying miRNA156, or a downstream gene target of the miRNA156, orthologues sequences in various tree crop species of interest.
Stage 2: Designing and synthesizing gRNA molecules corresponding to (or complementary to) the sequence targeted for editing, i.e. miRNA156. It is noted that the editing event is preferably targeted to a unique restriction site sequence to allow easier screening for plants carrying an editing event within their genome. According to some aspects of the invention, the nucleotide sequence of the gRNAs should be completely compatible with the genomic sequence of the target gene. Therefore, for example, suitable gRNA molecules should be constructed for different miRNA156, homologues of different tree crop strains.

The term ‘PAM’ refers hereinafter to Protospacer Adjacent Motif, which is a 2-6 base pair DNA sequence immediately following the DNA sequence targeted by the Cas nuclease in the CRISPR bacterial adaptive immune system.

The above gRNA molecules have been cloned into suitable vectors and their sequence has been verified. In addition different Cas (e.g. Cas9) versions have been analyzed for optimal compatibility between the Cas (e.g. Cas9) protein activity and the gRNA molecule in the specific tree crop plant.

The efficiency of the designed gRNA molecules have been validated by transiently transforming tissue culture of a selected tree crop species. A plasmid carrying a gRNA sequence together with the Cas9 gene has been transformed into protoplasts. The protoplast cells have been grown for a short period of time and then were analyzed for existence of genome editing events. The positive constructs have been subjected to the herein established stable transformation protocol into tree plant tissue for producing genome edited plants in miRNA156.

Stage 3: Transforming tree plants using Agrobacterium or biolistics (gene gun) methods. For Agrobacterium and biolistics, a DNA plasmid carrying (Cas9+gene specific gRNA) can be used. A vector containing a selection marker, Cas9 gene and relevant gene specific gRNA's is constructed. For biolistics, Ribonucleoprotein (RNP) complexes carrying (Cas9 protein+gene specific gRNA) are used. RNP complexes are created by mixing the Cas9 protein with relevant gene specific gRNA's.

According to some embodiments of the present invention, transformation of various tree crop tissues was performed using particle bombardment of:
DNA vectors
Ribonucleoprotein complex (RNP's)

According to further embodiments of the present invention, transformation of various tree crop tissues was performed using Agrobacterium (Agrobacterium tumefaciens) by:
Regeneration-based transformation
Floral-dip transformation
Seedling transformation
   Transformation efficiency by A. tumefaciens has been compared to the bombardment method by transient reporter gene expression. After transformation, reporter gene function of the transformants is verified.

According to further embodiments of the present invention, additional transformation tools were used in tree crops, including, but not limited to:
Protoplast PEG transformation
Extend RNP use
Directed editing screening using fluorescent tags
Electroporation

Selection of positive transformants is performed on DNA extracted from leaf sample of regenerated transformed plants and PCR is performed using primers flanking the edited region. PCR products are then digested with enzymes recognizing the restriction site near the original gRNA sequence. If editing event occurred, the restriction site will be disrupted and the PCR product will not be cleaved. No editing event will result in a cleaved PCR product.
Stage 4: Regeneration in tissue-culture. When transforming DNA constructs into the plant, antibiotics is used for selection of positive transformed plants. An improved regeneration protocol was herein established for the tree plant.
Stage 5: Selection of positive transformants. Once regenerated plants appear in tissue culture, DNA is extracted from leaf sample of the transformed plant and PCR is performed using primers flanking the edited region. PCR products are then digested with enzymes recognizing the restriction site near the original gRNA sequence. If editing event occurred, the restriction site will be disrupted and the PCR product will not be cleaved. No editing event will result in a cleaved PCR product.

Screening for CRISPR/Cas9 gene editing events has been performed by at least one of the following analysis methods:
Restriction Fragment Length Polymorphism (RFLP)
Next Generation Sequencing (NGS)
PCR fragment analysis
Fluorescent-tag based screening
High resolution melting curve analysis (HRMA)

The analysis of the digestion of the resulted PCR amplicon containing the gene specific gRNA sequence, by RNP complex containing Cas9, included the following steps:
1) Amplicon was isolated from the plant by primers flanking the sequence of the gene of interest targeted by the predesigned gRNA.
2) RNP complex was incubated with the isolated amplicon.
3) The reaction mix was then loaded on agarose gel to evaluate Cas9 cleavage activity at the target site.
Stage 6: Selection of transformed tree crop plants presenting prolonged adult or reproductive phase, and shortened juvenile phase, related phenotypes resulting from silencing of miRNA156.

It is within the scope that different gRNA promoters were tested in order to maximize editing efficiency.

It is within the scope that different gRNA promoters were tested in order to maximize editing efficiency.

The genome editing events herein described introduce mutations that silence or significantly reduce miRNA156, or upregulate downstream gene target of the miRNA156 expression or function in the plant.

By silencing miRNA156, or upregulating downstream gene target of the miRNA156 by gene editing technique, tree crops with earlier juvenility to reproductive phase transition are provided. Such modified tree crops produce enhanced yield (e.g. fruit or vegetables) in a shorter period of time. These edited miRNA156, or upregulated downstream gene target of the miRNA156 tree plants are highly desirable since their development and growth can be regulated. More specifically, the regularity of the juvenility to vegetative-reproductive switch is controlled to produce a high yield phenotype.

### Sequence Listing

The sequence listing was generated according to WIPO standard ST.26, wherein it is required to replace the “u” nucleotide by “t” in the RNA sequences.

### REFERENCES

1. Wu, G. et al. The Sequential Action of miR156 and miR172 Regulates Developmental Timing in Arabidopsis. Cell 138, 750-759 (2009).
2. Jia, X. L. et al. miR156 switches on vegetative phase change under the regulation of redox signals in apple seedlings. Sci. Rep. 7, 14223 (2017).
3. Ahsan, M. U. et al. Juvenility and Vegetative Phase Transition in Tropical/Subtropical Tree Crops. Front. Plant Sci. 10, 729 (2019).
4. Xie, K. and Yang Y. RNA-guided genome editing in plants using a CRISPR-Cas system. Molecular plant, 6 (6), 1975-1983 (2013).
5. Schwab, R. et al. Specific effects of microRNAs on the plant transcriptome. Dev Cell. 8(4), 517-27 (2005).
6. Xie, K. et al. Genomic Organization, Differential Expression, and Interaction of SQUAMOSA Promoter-Binding-Like Transcription Factors and microRNA156 in Rice. Plant Physiology, 142 (1), 280-293 (2006).
7. Fu, C. et al. Overexpression of miR156 in switchgrass (Panicum virgatum L.) results in various morphological alterations and leads to improved biomass production. Plant Biotechnol J. 10(4): 443-452 (2012).
8. Chuck, G. S., et al. Overexpression of the maize Corngrass1 microRNA prevents flowering, improves digestibility, and increases starch content of switchgrass. PNAS 108 (42), 17550-17555 (2011).
9. Tatematsu, K. et al. A molecular mechanism that confines the activity pattern of miR165 in Arabidopsis leaf primordia. The Plant Journal 82(4), 596-608 (2015).
10. Reinhart, B. J. et al. MicroRNAs in plants. Genes Dev. 16, 1616-1626 (2002).
11. Xing, S. et al. miR156-Targeted and Nontargeted SBP-Box Transcription Factors Act in Concert to Secure Male Fertility in Arabidopsis. Plant Cell 22, 3935-3950 (2010).
12. Jiao, Y. et al., Regulation of OsSPL14 by OsmiR156 defines ideal plant architecture in rice. Nat. Genetics. 42, 541-544 (2010).
13. Miura, K. et al., OsSPL14 promotes panicle branching and higher grain productivity in rice. Nat. Genetics. 42, 545-549 (2010).
14. Nonogaki, H. MicroRNA Gene Regulation Cascades During Early Stages of Plant Development. Plant Cell Physiol. 51, 1840-1846 (2010).

1. A method for controlling juvenile to adult phase transition in a tree plant, the method comprising introducing using targeted genome editing, at least one genomic modification in an endogenous microRNA156 (miRNA156).

2. The method according to claim 1, wherein the method comprises upregulation of a downstream gene target of the miRNA156.

3. The method according to claim 1, wherein the method comprises expressing in the tree plant a nuclease comprising a nucleic acid binding domain, wherein the nucleic acid binding domain binds to a target site in the miRNA156, to generate the genomic modification.

4. (canceled)

5. The method according to claim 3, wherein the nuclease introduces a mutation in the miRNA156 via a DNA cleavage domain that cleaves the miRNA156 encoding gene, or via base editing of the miRNA156 encoding gene.

6. (canceled)

7. The method according to claim 1, wherein said genomic modification is generated via introduction of (a) Cas DNA or a nuclease-deactivated (dCas) and gRNA polynucleotide comprising a nucleic acid sequence complementary to a target site in the miRNA156, or (b) a ribonucleoprotein (RNP) complex comprising Cas protein and gRNA polynucleotide comprising a nucleic acid sequence complementary to a target site in the miRNA156, and any combination thereof.

8. (canceled)

9. (canceled)

10. (canceled)

11. The method according to claim 1, wherein said genomic modification is a silencing mutation, a knockdown mutation, a knockout mutation, a loss of function mutation or any combination thereof.

12. (canceled)

13. (canceled)

14. The method according to claim 1, wherein said method comprises reducing the duration of juvenile phase in the tree plant by introduction of a targeted genomic modification in the microRNA156 (miRNA156), thereby down regulating expression of the endogenous microRNA156 (miRNA156) and/or upregulating of a downstream gene target of the miRNA156, wherein the downstream gene target of the miRNA156 is selected from genes comprising a miR156 recognition site or element.

15. (canceled)

16. The method according to claim 14, wherein the downstream gene target of the miRNA156 is selected from genes encoding SQUAMOSA PROMOTER BINDING PROTEIN (SBP)-domain containing transcription factors, SQUAMOSA PROMOTER BINDING PROTEIN-LIKE (SPL) transcription factors and SUPPRESSOR OF OVEREXPRESSION OF CONSTANS 1 (SOC1).

17. (canceled)

18. (canceled)

19. The method according to claim 1, wherein the tree crop exhibits time reduced juvenility or vegetative phase change, increased duration of adult or mature phase, early transition into adult or mature or reproductive phase, enhanced productivity trait (fruit or vegetable yield), enhanced agronomic trait and/or enhanced plant biomass, relative to a corresponding tree plant lacking the targeted genomic modification, and/or an altered trait selected from the group consisting of: an altered proportion of juvenile, transitional, or adult leaves; enhanced yield of vegetative tissue; enhanced fruit yield; and altered development or morphology; wherein the trait exhibited by the tree plant is altered relative to a corresponding wild type tree plant.

20. The method according to claim 1, wherein the method comprises modulating the timing of juvenile to adult phase transition, wherein the timing of juvenile to adult phase transition is calculated based on miRNA156 target genes expression level and is shorter compared to wild type non edited corresponding plants.

21. (canceled)

22. The method according to claim 1, wherein the method comprises using targeted genome editing, generating at least one genomic modification in an endogenous microRNA156 (miRNA156) and/or upregulating a downstream gene target of the miRNA156.

23. A tree plant, plant cell, plant tissue, plant part or a seed, produced by the method of claim 1.

24. A tree plant exhibiting modulated timing of juvenile to adult phase transition, wherein the tree plant comprises introduced targeted genome modification in a microRNA156 (miRNA156), and/or upregulated expression of a downstream gene target of the miRNA156.

25.-33. (canceled)

34. The tree plant according to claim 24, wherein said genomic modification is a silencing mutation, a knockdown mutation, a knockout mutation, a loss of function mutation or any combination thereof.

35. (canceled)

36. (canceled)

37. The tree plant according to claim 24, wherein said tree plant exhibits reduced duration of the juvenile phase, time reduced juvenility or vegetative phase change, increased duration of adult or mature phase, and/or early transition into adult or mature or reproductive phase, relative to a corresponding tree plant lacking the targeted genomic modification, further wherein the plant comprises a targeted genomic modification in the microRNA156 (miRNA156), said modification down regulate expression of the endogenous microRNA156 (miRNA156) and/or upregulate expression of a downstream gene target of the miRNA156, wherein the downstream gene target of the miRNA156 is selected from genes comprising a miR156 recognition site or element.

38. (canceled)

39. The tree plant according to claim 37, wherein the downstream gene target of the miRNA156 is selected from genes encoding SQUAMOSA PROMOTER BINDING PROTEIN (SBP)-domain containing transcription factors, SQUAMOSA PROMOTER BINDING PROTEIN-LIKE (SPL) transcription factors and SUPPRESSOR OF OVEREXPRESSION OF CONSTANS 1 (SOC1).

40. (canceled)

41. (canceled)

42. (canceled)

43. The tree plant according to claim 24, wherein the tree plant exhibits an altered trait selected from the group consisting of: an altered proportion of juvenile, transitional, or adult leaves; enhanced yield of vegetative tissue; enhanced fruit yield, altered development or morphology, wherein the trait exhibited by the tree plant is altered relative to a corresponding wild type tree plant and/or wherein the timing of juvenile to adult phase transition is shorter compared to wild type non edited corresponding plants.

44. A plant cell, plant tissue, regenerable cells, plant part or a seed of the tree plant according to claim 24.

45. A polynucleotide molecule comprising a promoter operably linked to a nucleotide sequence encoding a Cas nuclease and a guide RNA (gRNA) comprising a nucleic acid sequence complementary to a target site in miRNA156.

46. The polynucleotide molecule according to claim 45, wherein the sequence of the miRNA156 is selected from SEQ ID NO:1-6, a functional variant thereof or any combination thereof.

47. (canceled)

48. (canceled)
